(19) United States
(12) Patent Application Publication
(10) Pub. No.: US 2019/0290762 A1
(43) Pub. Date: Sep. 26, 2019

(54) NANO-ARCHITECTURED COLLOIDOSOMES FOR CONTROLLED AND TRIGGERED RELEASE

(71) Applicants: Nitin CHOPRA, Sugar Land, TX (US); Peter Hendrikus Theodorus VOLLENBERG, Geleen (NL); Ihab N. ODEH, Sugar Land, TX (US); C. William GUNDLACH, VII, Sugar Land, TX (US); Jonathon SCHOLIN, Sugar Land, TX (US); SABIC Global Technologies B.V., Bergen op Zoom (NL)

(72) Inventors: Jonathon Scholin, Sugar Land, TX (US); Sidney Vastenhout, Geleen (NL); Peter Hendrikus Theodorus Vollenberg, Geleen (NL); Nitin Chopra, Sugar Land, TX (US); Ihab N. Odeh, Sugar Land, TX (US); C. William Gundlach, IV, Sugar Land, TX (US)

(21) Appl. No.: 16/302,843

(22) PCT Filed: May 31, 2017
(86) PCT No.: PCT/IB2017/053207

§ 371 Date: Nov. 19, 2018

Related U.S. Application Data
(60) Provisional application No. 62/349,500, filed on Jun. 13, 2016.
(60) Provisional application No. 62/510,343, filed on May 24, 2017.

(51) Int. Cl. A61K 41/00; A61K 47/30; A61K 9/127; A61K 9/50

(52) U.S. Cl. CPC A61K 41/0028; A61K 47/30; B82Y 5/00; A61K 9/5089; A61K 9/127

(57) ABSTRACT

Colloidosome containing active agents and uses thereof are described. The colloidosome can include (a) a responsive micro- or nanostructured porous shell defined by a plurality of nanomaterials and interstices formed between the micro- or nanomaterials and (b) a core that is defined by the responsive micro- or nanostructured porous shell. The shell is loaded with an active agent capable of being released from the shell in response to a stimulus.

### CROSS REFERENCE TO RELATED APPLICATIONS

This application claims the benefit of priority of U.S. Provisional Patent Application No. 62/349,500 filed Jun. 13, 2016, and U.S. Provisional Patent Application No. 62/510,343 filed May 24, 2017. The entire contents of each of the above-referenced disclosures are specifically incorporated herein by reference without disclaimer.

### BACKGROUND OF THE INVENTION

### A. Field of the Invention

The invention generally concerns a colloidosome that includes a responsive nanostructured porous shell defined by a plurality of nanomaterials and interstices formed between the nanomaterials. The colloidosome shell can be loaded with an active agent that is capable of being released from the shell in response to a stimulus.

### B. Description of Related Art

Nanostructured encapsulation systems (e.g., colloidosomes, encapsulated nanoparticles, liposomes, and the like) can provide improved uptake and efficient transport or delivery of active agents to intended targets (e.g., a person, an animal, an inanimate object, etc.). Colloidosomes are typically formed such that the core is loaded with the active agent. Release of flavors, fragrance, air fresheners, lotions, creams, nutrients, textile scents, drugs, etc., have been released from such colloidosome cores. (See, Ajazzuddin et al., Recent Patents on Nanomedicine, 2015, 5, 3-11.)

U.S. Patent Application Publication No. 20040096515 to Bausch et al. describes a method for making self-assembled, selectively permeable colloidosomes that have controlled pore-size, porosity, and mechanical properties. Sander et al. (Langmuir, 2013, 29(49), pp. 15168-15173) describes encapsulating functional nanoparticles within a colloidosome hollow core. The nanoparticles can be released from the core by either swelling or rupture of the colloidosome shell. U.S. Patent Application No. 2013/0316008 to Nallani et al. describes a multi-compartmentalized vesicular structure loaded with active compounds. The active compounds can be released from the cores of the multi-compartmentalized structure. U.S. Patent Application No. 2015/0368407 to Zhang et al. describes nested capsules suitable for delivering and selectively releasing an encapsulate at a targeted location.

Such core loaded colloidosome systems, however, have several limitations. By way of example, these systems tend to have a more spike or burst release profile of the active agent rather than a more controlled and tunable release profile. Further, it is difficult to load multiple active agents into the core or in nested structures and prevent having the agents chemically react or mix with one another. Additionally, the loading capacity of the colloidosomes are limited by their core volumes and the composition of the shells can impose thermodynamic limits on the type of actives to be stored. Also, the types of stimuli that can be used to release active agents from the core is limited.

### SUMMARY OF THE INVENTION

A discovery has been made that solves the aforementioned problems associated with nanostructured colloidosome systems having actives loaded within their cores. The solution resides in a micro- or nanostructured colloidosome that includes a responsive micro- or nanostructured porous shell having an active agent(s) loaded therein. In particular, the shell can be defined by a plurality of micro- or nanomaterials and interstices formed between the nanomaterials. Active agents can be loaded into the micro- or nanomaterials, on the surface of the micro- or nanomaterials, and/or within the interstices of the micro- or nanomaterials, or any combination thereof. This loading structure and capacity of the colloidosome shell provides for a variety of possible release mechanisms in response to a variety of stimuli (e.g., pH range, electromagnetic radiation, a temperature range, a mechanical force, humidity, the presence or absence of a chemical substance, an odor, or any combination thereof). The shell can have a single or multi-shell (e.g., layered) colloidosome architecture, where each shell or layer can be loaded with an active agent or multiple active agents. In contrast to conventional colloidosomes, where the core is the only compartment used for storing an active agent, the nanostructured shell of the present invention expands on the possibilities for active agent loading options and ultimately for triggered and controllable release responses. Stated another way, the nanostructured colloidosomes of the present invention can have an increased loading efficiency (with respect to active agents via the use of the shell) and multiple release profiles in response to various stimuli, thereby allowing for a wide range of possibilities to meet a variety of needs or objectives.

Additionally, the colloidosome architecture of the present invention can enable size-dependent light-matter and/or opal-like phenomena due to the micro- or nanoscale components and porosity of the shell or differences in refractive index of the matter compositions and the interfaces present. The methods of making the micro- or nanomaterials described throughout the specification can facilitate tunability of the colloidosomes, which can be helpful in intensifying or narrowing the spectrum for light-matter interactions. This allows for a variety of colors that can be imparted to a composition having the colloidosomes of the present invention.

In one aspect of the present invention, a colloidosome can include a responsive micro- or nanostructured porous shell defined by a plurality of micro- or nanomaterials and interstices formed between the micro- or nanomaterials, and a core that is defined by the responsive micro- or nanostructured porous shell. The shell can be loaded with one or more active agents and can be capable of releasing one or more active agents (e.g., 2, 3, 4, 5, 6, 7, etc. active agents) from the shell in response to the same or different stimuli. Release of the active agent from the shell can be done through expansion, contraction, constriction, reaction, folding, or dissolution of the shell in response to the stimulus or stimuli.

The plurality of micro- or nanomaterials that define the shell can be porous and/or hollow structures. By way of example, hollow and/or porous micro- or nanogel particles, polymer brushes, surfactant molecules, hollow and/or porous metal oxide particles, lipid particles, block copolymers, biopolymers, biomolecules, micellar/dendrimer structures, yolk/shell structures, core/shell structures, pomegranate structures, hollow or porous structures/nanomaterials, functionalized hollow or porous micro- or nanostructures, aerogels, or combinations thereof. In one instance, the plurality of micro- or nanomaterials are micro- or nanogel particles having a gel phase that include a polymer network of hydrophilic, hydrophobic, amphiphilic, amphiphobic, lipophilic, or lipophobic polymers, or a combination thereof. In a preferred embodiment, the polymer network is cross-linked. Such polymer networks can include polyvinyl alcohol (PVA), poly(N-isopropyl acrylamide) (pNIPAAm), cross-linked N-isopropylacrylamide, functionalized polyNIPAAm (e.g., CO₂H-NIPAAm, NH₂-NIPAAm, etc.), copolymers of NIPAAm and N,N′-methylenebisacrylamide (MBA), co-polymers of NIPAAm and allyl amine, copolymers of NIPAAm and acrylic acid, poly(ethylene glycol), a hydroxylated poly(methyl methacrylate), an ethylene-vinyl acetate copolymer, 2-hydroxyethyl methacrylate (HEMA), poly (maleic acid/octyl vinyl ether) (PMAOVE), a polyurethane, poly(acrylic acid), poly(stearyl acrylate) (PSA), poly(acrylamide) and copolymers thereof such as dipropylene glycol acrylate caprylate (DGAC) or dipropylene glycol diacrylate sebacate (DGDS) (a cross-linker), starch, chitin or a derivative thereof, silicone or a derivative thereof, or a polyolefin, co-polymers of N-isopropylacrylamide and glutaraldehyde, or any combination thereof. The polymeric network can include cationic, anionic, or zwitterionic polymers or polymers that include metal-organic frameworks or zeolitic imidazolate frameworks. Formation of the colloidosome can occur by attaching the plurality of micro- or nanomaterials to one another. Such attachment can occur through a chemical bond, electrostatic interaction, van der Waals interaction, ionic interaction, hydrogen bonding, dipolar interaction, or any combination thereof. In some embodiments, colloidosomes are formed by cross-linking microstructures, nanostructures, or both together. By way of example, glutaraldehyde and/or 1,4-butanediol diglycidyl ether can be used to crosslink micro- or nanostructures formed from NH₂-functionalized pNIPAAm. The micro- or nanostructured architecture of the shell provides for superior mechanical properties (e.g., a yield strength of 1 kPa to 1 MPa or 1 kPa to 50 kPa). In some instances, the shell can include a second responsive micro- or nanostructured porous shell that encompasses the first shell and includes a second set of a plurality of micro- or nanomaterials and interstices formed between the second set of micro- or nanomaterials. The core of the colloidosome can be a polymer emulsion, a polymer gel, an aerogel, a liquid (e.g. an oil), or a void space. An overall size of the colloidosome can range from 5 to 30000 nm, with the average size of the plurality of micro- or nanomaterials being 2 nm to 15000 nm, and/or the size of the core being 5 nm to 2500 nm, with the proviso that the size of the core is larger than the average size of the plurality of micro- or nanomaterials that make up the shell. In a preferred aspect, the polymer matrix includes poly(N-isopropyl acrylamide) (pNIPAAm), functionalized polyNIPAAm (e.g., CO₂H-NIPAAm, NH₂-NIPAAm, etc.), cross-linked NIPAAm copolymers of NIPAAm and N,N′-methylenebisacrylamide (MBA) or combinations thereof.

In some instances, the gel can be an aerogel. Non-limiting examples of aerogels include a silicon dioxide aerogel or an organic polymer aerogel, a carbon aerogel, or the like. The porosity and particle size that are critical for the release characteristics can be tuned by the selection of process parameters conditions (e.g., acid-base catalysis, surfactants, type of silicon precursors, temperature, time, and mixing conditions) employed to make the aerogel. An aerogel is an open-celled, mesoporous, solid foam that is composed of a network of interconnected nanostructures and that exhibits a porosity (non-solid volume) of no less than 80%. The pores have a diameter in the range of <1 to 100 nanometers and usually <20 nm. The high surface area and open-porous skeleton of the aerogel can potentially serve to encapsulate the active.

The active agents can be bound to the surface of the plurality of micro- or nanomaterials, loaded or impregnated within the plurality of micro- or nanomaterials, or contained within the plurality of interstices formed between the micro- or nanomaterials. In some instances, the core can also be loaded with one or more active agents. In other instances, the core may not have any active agents loaded therein. Non-limiting examples of the active agents that can be used in the context of the present invention include chemical agents, biological agents, oils, ionic liquids, suspensions, polymers, or any combination thereof. Chemical agents can include a drug, gaseous molecules, a cosmetic agent, a flavoring agent, a fragrance-producing chemical, a malodor agent, a reactive agent, a cross-linker, a reactive diluent, a solvent, an inorganic or organic chemical, a organometallic system, a petrochemical, a reducing or oxidizing agent, or an aqueous salt, or any combination thereof. The biological agent can include a protein, a peptide, a nucleic acid, a carbohydrate, a lipid, or any combination thereof.

The colloidosomes of the present invention can be used in a variety of methods and applications. By way of example, one method can include subjecting the colloidosome to a stimulus to release and deliver the active agent. In some instances, the active agent can be controllably released from the colloidosome. The colloidosome can be incorporated into a variety of compositions. Non-limiting examples of such compositions include pharmaceutical compositions, cosmetic compositions, personal care products, fragrances, perfumes, compositions intended for inanimate objects or surfaces (e.g., cleansers, disinfectants, dish detergents, laundry detergents), etc. The colloidosomes can be used in a variety of applications ranging from drug delivery, catalysis, nanocomposites, bioanalysis, diagnostics, sensors and markers, energy storage, bio-inhibitors (repellants pesticides, herbicides), urea release, self-repair (paints, paper, textile, concrete, etc.), flame retardants, personal care (skin care, fragrances or perfumes, hair, teeth, etc.), nutritional additives, vitamins, flavors, pigments, textile scent, detergents, softeners, animal care, lubricants, adhesives, etc.

Methods of making the colloidosomes of the present invention are also described. One method can include (a) obtaining a plurality of micro- or nanomaterials and a cross-linking agent, (b) obtaining an active agent, and (c) forming a responsive micro- or nanostructured porous shell from the micro- or nanomaterials by combining the micro- or nanomaterials, cross-linking agent, and the active agent in a liquid medium (e.g., aqueous medium). The liquid medium can be mixed or moved through a membrane to obtain the colloidosome. The liquid can also be mixed using emulsification techniques, for example, vortexing, sonicating, or shaking at high revolutions per minute (rpm). An active agent can be (i) bound to the surface of the plurality of nanomaterials or loaded or impregnated within the plurality of micro- or nanomaterials from step (a) and/or (ii) present in the liquid medium. In some embodiments, the colloidosomes can be made in the absence of alcohol and/or emulsifiers.

In one aspect of the invention, 20 embodiments are described. Embodiment 1 is a colloidosome comprising (a) a responsive micro- or nanostructured porous shell defined by a plurality of micro- or nanomaterials and interstices formed between the micro- or nanomaterials, wherein the shell is loaded with an active agent that is capable of being released from the shell in response to a stimulus; and (b) a core that is defined by the responsive micro- or nanostructured porous shell. Embodiment 2 is the colloidosome of embodiment 1, wherein the plurality of micro- or nanomaterials are microgel particles, nanogel particles, polymer brushes, surfactant molecules, metal oxide particles, lipid particles, block copolymers, cross-linked polymers, biopolymers, biomolecules, micellular/dendrimer structures, yolk/shell structures, core/shell structures, pomegranate structures, hollow structures/nanomaterials, functionalized micro- or nanostructures, or combinations thereof. Embodiment 3 is the colloidosome of embodiment 2, wherein the plurality of nanomaterials are micro- or nanogel particles having a gel phase comprising a polymer network of hydrophilic, hydrophobic, amphiphilic, amphiphobic, lipophilic or lipophobic polymers, or a combination thereof. Embodiment 4 is the colloidosome of embodiment 3, wherein the polymer network comprises a crosslinked polymer or co-polymer and wherein the polymer comprise poly(N-isopropylacrylamide) (pNIPAAm), pNIPAAm-poly(N,N′-methylenebisacrylamide) copolymer, poly(ethylene glycol), functionalized (pNIPAAm), polyvinyl alcohol (PVA), a hydroxylated poly(meth)acrylate, an ethylene-vinyl acetate copolymer, 2-hydroxyethyl methacrylate (HEMA), poly (maleic acid/octyl vinyl ether) (PMAOVE), a polyurethane, poly(acrylic acid), poly(stearyl acrylate) (PSA), poly(acrylamide) and copolymers thereof; or a polyolefin, or any combination thereof. Embodiment 5 is the colloidosome of any one of embodiments 1 to 4, wherein the stimulus is a temperature range, a pH range, electromagnetic radiation, a mechanical force, humidity, the presence or absence of a chemical substance, an odor, or any combination thereof. Embodiment 6 is the colloidosome of any one of embodiments 1 to 5, wherein the shell has a yield strength of 1 kPa to 1 MPa or 1 kPa to 50 kPa. Embodiment 7 is the colloidosome of any one of embodiments 1 to 6, wherein the active agent is bound to the surface of the plurality of micro- or nanomaterials, loaded or impregnated within the plurality of micro- or nanomaterials, or contained within the plurality of interstices formed between the micro- or nanomaterials. Embodiment 8 is the colloidosome of embodiment 7, further comprising a second active agent. Embodiment 9 is the colloidosome of embodiment 8, wherein the active agent and the second active agent are different and optionally are capable of reacting with one another upon their release from the shell to form an activated material. Embodiment 10 is the colloidosome of any one of embodiments 8 to 9, wherein the shell is capable of releasing the active agent and the second active agent in response to the same or different stimuli. Embodiment 11 is the colloidosome of any one of embodiments 1 to 10, further comprising a second responsive micro- or nanostructured porous shell that encompasses the shell, wherein the second shell includes a second set of a plurality of micro- or nanomaterials and interstices formed between the second set of micro- or nanomaterials. Embodiment 12 is the colloidosome of any one of embodiments 1 to 11, wherein the core is a polymer emulsion or a polymer gel, or a void space, and wherein the core is optionally loaded with a core active agent. Embodiment 13 is the colloidosome of any one of embodiments 1 to 12, wherein the overall size of the colloidosome is 5 to 30000 nm, the average size of the plurality of micro- or nanomaterials is 2 nm to 15000 nm, and/or the size of the core is 5 nm to 2500 nm, with the proviso that the size of the core is larger than the average size of the plurality of micro- or nanomaterials. Embodiment 14 is the colloidosome of any one of embodiments 1 to 13, wherein the active agent and/or the core active agent is a chemical agent, a biological agent, an oil, an ionic liquid, a suspension, or a polymer, or any combination thereof. Embodiment 15 is the colloidosome of embodiment 14, wherein: the chemical agent is a drug, a cosmetic agent, a flavoring agent, a fragrance-producing chemical, a malodor agent, a reactive agent, a cross-linker, a reactive diluent, a solvent, an inorganic or organic chemical, a metallo-organic system, a petrochemical, a reducing or oxidizing agent, or an aqueous salt, or any combination thereof; and/or the biological agent is a protein, a peptide, a nucleic acid, a carbohydrate, a lipid, or any combination thereof. Embodiment 16 is the colloidosome of any one of embodiments 1 to 15, wherein the plurality of micro- or nanomaterials are attached to one another by a chemical bond, electrostatic interaction, van der Waals interaction, ionic interaction, hydrogen bonding, dipolar interaction or any combination thereof. Embodiment 17 is the colloidosome of any one of embodiments 1 to 16, wherein the colloidosome is comprised in a pharmaceutical composition, a topical skin care composition, an optically clear medium, or a composition intended to be applied to an inanimate object.

Embodiment 18 is a method of using any one of the colloidosomes of embodiments 1 to 17 to deliver an active agent, the method comprising subjecting the colloidosome to a stimulus to release and deliver the active agent. Embodiment 19 is the method of embodiment 18, wherein the active agent is controllably released from the colloidosome. Embodiment 20 is a method of making any one of the colloidosomes of embodiments 1 to 19, the method comprising: (a) obtaining a first solution comprising micro- or nanomaterials and a cross-linking agent comprising glutaraldehyde or 1,4-butanediol diglycidyl ether; (b) obtaining a second solution comprising an active agent; and (c) combining the solutions to form a colloidosome comprising a micro- or nanostructured porous shell comprising a cross-linked polymeric network; wherein an active agent is (i) bound to the surface of the micro- or nanostructured porous shell, or loaded or impregnated within the shell of step and/or (ii) present in the liquid medium.

The following includes definitions of various terms and phrases used throughout this specification.

The term “colloidosome” refers to a structure that has a shell defined by a plurality of nanomaterials and interstices formed between the nanomaterials and a core that is defined by the nanostructure shell. The nanomaterials are attached to one another by a covalent bond, electrostatic interaction, van der Waals interaction, ionic interaction, hydrogen bonding, dipolar interaction, or any combination thereof. The core can be void (e.g., empty) or include other materials, compounds, or structures. Non-limiting examples of a colloidosomes of the present invention are illustrated in FIGS. 1A through 1D.

“Nanomaterial” or “nanostructure” refers to an object or structure in which at least one dimension of the object or structure is equal to or less than 1000 nm (e.g., one dimension is 1 to 1000 nm in size). In a particular aspect, the nanomaterial includes at least two dimensions that are equal to or less than 1000 nm (e.g., a first dimension is 1 to 1000 nm in size and a second dimension is 1 to 1000 nm in size). In another aspect, the nanomaterial includes three dimensions that are equal to or less than 1000 nm (e.g., a first dimension is 1 to 1000 nm in size, a second dimension is 1 to 1000 nm in size, and a third dimension is 1 to 1000 nm in size). The shape of the nanomaterial can be of a wire, a particle (e.g., having a substantially spherical shape), a rod, a tetrapod, a hyper-branched structure, a tube, a cube, or mixtures thereof. Nanomaterials can be nanogel particles, polymer brushes, surfactant molecules, metal oxide particles, lipid particles, block copolymers, biopolymers, biomolecules, micellular/dendrimer structures, yolk/shell structures, core/shell structures, pomegranate structures, hollow structures/nanomaterials, functionalized nanostructures, aerogels, or combinations thereof.

“Micromaterial” or “microstructure” refers to an object or structure in which at least one dimension of the object or structure is greater than 1000 nm and up to 100,000 nm (e.g., one dimension is 1001 to 100,000 nm in size). In a particular aspect, the micromaterial includes at least two dimensions that are greater than 1000 nm or up to 100,000 nm (e.g., a first dimension is 1001 to 100,000 nm in size and a second dimension is 1001 to 100,000 nm in size). In another aspect, the micromaterial includes three dimensions that are greater than 1000 nm or up to 100,000 nm (e.g., a first dimension is 1001 to 100,000 nm in size, a second dimension is 1 to 1000 nm in size, and a third dimension is 1001 to 100,000 nm in size). The shape of the micromaterial can be of a wire, a particle (e.g., having a substantially spherical shape), a rod, a tetrapod, a hyper-branched structure, a tube, a cube, or mixtures thereof. Micromaterials can be microgel particles, polymer brushes, surfactant molecules, metal oxide particles, lipid particles, block copolymers, biopolymers, biomolecules, micellular/dendrimer structures, yolk/shell structures, core/shell structures, pomegranate structures, hollow structures/micromaterial s, functionalized microstructures, aerogels, or combinations thereof.

The “self-assembly” phrase refers to the ability of the nanomaterials to come together and interact to form a colloidosome in a liquid composition (e.g., aqueous solution). FIGS. 1A-D, provides a non-limiting illustration of a plurality of nanomaterials 106 that have self-assembled to form a shell 102.

The “core/shell” phrase encompasses both core/shell and yolk/shell structures, with the difference being that in a core/shell structure at least 50% of the surface of the “core” contacts the shell. By comparison, a yolk/shell structure includes instances where less than 50% of the surface of the “yolk” contacts the shell.

Determination of whether a core, yolk, or void space is present in the core/shell structures or materials of the present invention can be made by persons of ordinary skill in the art. One example is visual inspection of a transition electron microscope (TEM) or a scanning transmission electron microscope (STEM) image of a core/graphene based shell structure or material of the present invention and determining whether a void space is present or determining whether at least 50% (core) or less (yolk) of the surface of a given active agent contacts the colloidosome shell or the nanomaterial shell.

The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The colloidosomes of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the colloidosomes of the present invention is their ability to contain and release active agents from their shell in response to a stimulus or multiple stimuli.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1A is an illustration of a nano-architectured colloidosome of the present invention.

FIG. 1B is an illustration of a nano-architectured colloidosome of the present invention having at least one active agent in the core of the colloidosome.

FIG. 1C is an illustration of a nano-architectured colloidosome of the present invention that includes a shell having two layers in contact with each other.

FIG. 1D is an illustration of a nano-architectured colloidosome of the present invention that includes a shell having two separated layers.

FIG. 2A is a cross-sectional illustration of a core/shell type nanomaterial of the present invention.

FIG. 2B is a cross-sectional illustration of another core/shell type nanomaterial of the present invention.

FIG. 2C is a cross-sectional illustration of a multiple yolk/shell type nanomaterial of the present invention.

FIG. 2D is a cross-sectional illustration of a core/shell type nanomaterial of the present invention having a shell with void space between an inner layer and an outer layer.

FIG. 2E is a cross-sectional illustration of a core/shell type nanomaterial of the present invention having two shell with two layers.

FIG. 2F is a cross-sectional illustration of multiple active agents distributed in a polymer matrix of the nanomaterial.

FIG. 3A is an illustration of release of active agents from a colloidosome of the present invention when subjected to a stimulus.

FIG. 3B is an illustration of a method of releasing multiple active agents from a colloidosome of the present invention when subjected to a stimulus.

FIG. 3C is an illustration of a method of releasing active agents from a colloidosome of the present invention when subjected to multiple stimuli.

FIG. 3D is an illustration of a method of releasing active agents from a colloidosome of the present invention by bursting an inner layer of the colloidosome.

FIG. 4A shows prophetic release profiles of a colloidosome of the present invention having the nanomaterial loaded with active agents (active agent A), active agents loaded in the interstices (active agent B), and active agents loaded in the core (active agent C).

FIG. 4B shows prophetic release profiles of a multi-shell (two-layer) colloidosome of the present invention having a first colloidosome shell loaded with an active agent (active agent A), active agents loaded in the interstices (active agent B) and core (active agent C), and a second colloidosome shell loaded with an active agent (active agent D).

FIG. 4C illustrates release profiles of a multi-shell (three-layer) colloidosome of the present invention having a first colloidosome shell loaded with an active (active agent A), an active agent (active agent B) loaded in the interstices, an active agent (active agent C) loaded in the core, a second colloidosome shell loaded with an active agent (active agent D), and a third colloidosome shell loaded with an active agent (active agent E).

FIG. 5 illustrates release profiles of a single shell colloidosome of the present invention having the nanomaterial loaded with multiple active agents (active agents A and D) loaded, an active agent (active agent B) loaded in the interstices, and an active agent (active agent C) loaded in the core.

FIGS. 6A-B show the optical images of swollen microgels prior to aging (FIG. 6A) and after aging (FIG. 6B).

FIG. 7A-E show images of the colloidosome structures of the present invention.

FIG. 8 is an optical image of a mixture of colloidosomes of the present invention of various sizes.

FIG. 9A shows an optical image of swollen NIPAAm microgels of the present invention.

FIG. 9B shows an optical image of colloidosomes of the present invention having a cross-linked microgel shell and a limonene core.

FIG. 10 is the average diameter size distribution data for the colloidosome of FIG. 9B.

FIG. 11 shows the relationship of particle-diameter (nm) to distribution by intensity for microgels (peak at about 100 nm) and colloidosomes (peak at about 915).

FIG. 12 shows an optical image of a mixture of colloidosomes of the present invention, swollen microgels and free limonene droplets.

FIG. 13 shows an optical image of colloidosomes of the present invention having different shapes.

FIG. 14 shows the relationship of limonene concentration over time of the colloidosomes of the present invention and a control.

FIG. 15 shows the relationship of released limonene from colloidosomes of the present invention versus temperature.

FIG. 16 shows the amount of limonene released over time for colloidosomes of the present invention in water (top line) and in a composition (bottom line).

FIG. 17 shows the relationship between percent transmission and concentration of colloidosomes of the present invention.

FIG. 18 shows an optical image of a control colloidosome absent a cross-linking agent.

While the invention is susceptible to various modifications and alternative forms, specific embodiments thereof are shown by way of example in the drawings and may herein be described in detail. The drawings may not be to scale.

### DETAILED DESCRIPTION OF THE INVENTION

A solution to the problems associated with controllable release of active agents in response to a stimulus or multiple stimuli has been discovered. The solution is premised on loading active agent(s) into the shells or shells and cores of the colloidosomes of the present invention. This is in contrast to conventional controlled release colloidosomes or nested capsules, both of which encapsulate the active agent in the core of the colloidosomes or capsules. The solution provides an elegant way to allow for tuning of a colloidosome for one or more specific applications. By way of example, the colloidosome shell can be tuned to allow for one or more kind of triggered-release mechanism(s) such as pH, temperature, light, vapor pressure or odor, light, humidity, mechanical force, and/or chemical environment (e.g., biomarkers, sweat, salt/electrolyte gradient, etc.) and/or one or more kind of storage systems of one or more active agents. The colloidosome size can be tuned to adjust the packing density and interstices between the packed nanostructures that make up the colloidosome.

These and other non-limiting aspects of the present invention are discussed in further detail in the following sections with reference to the Figures.

### A. Colloidosomes

The colloidosome of the present invention can include a responsive micro- or nanostructured porous shell loaded with an active agent that is capable of being released from the shell and a core (inner area). The shell is defined by a plurality of micro- or nanomaterials and interstices formed between the micro- or nanomaterials, and the core is defined by the responsive micro- or nanostructured porous shell. The colloidosomes can have multi-layers or shells and/or multiple active agents.

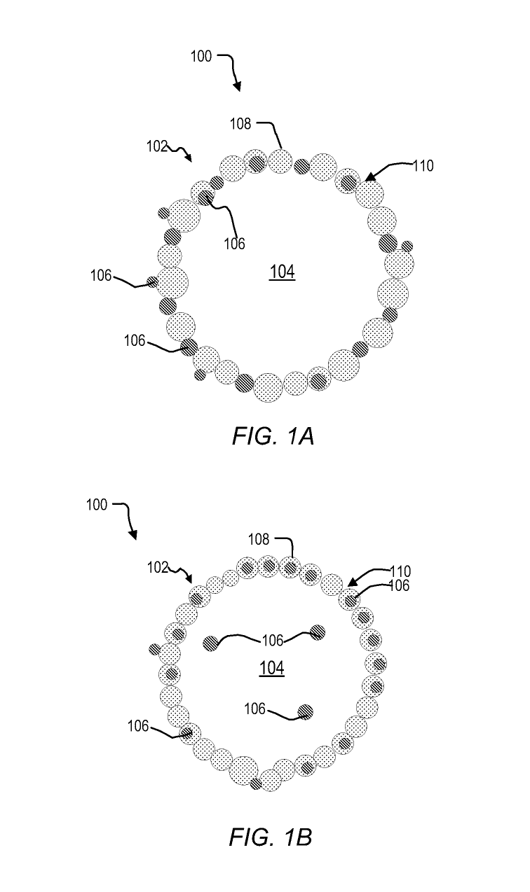

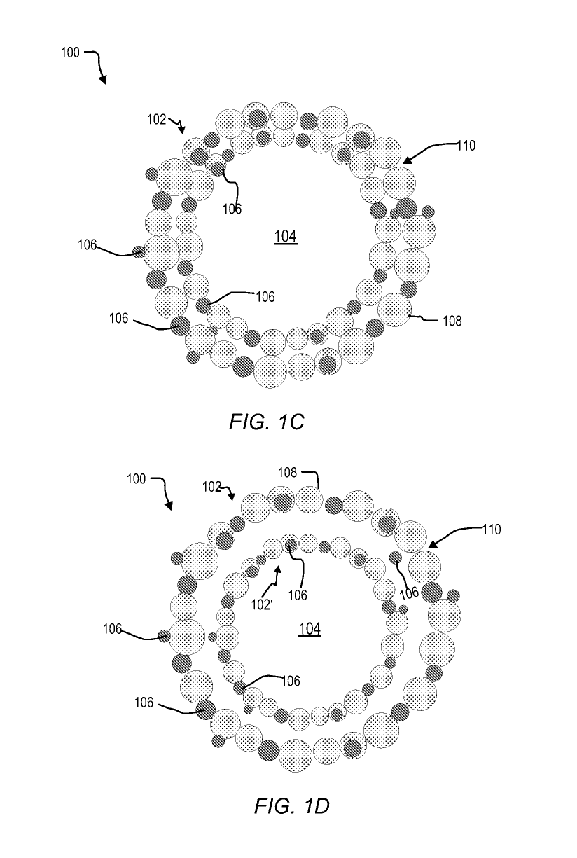

FIGS. 1A through 1D depict illustrations of colloidosomes 100 of the present invention. FIGS. 1A and 1B depict colloidosomes 100 that include a single shell colloidosome. FIG. 1B, also depicts colloidosome 100 having additional active agents loaded in the core of the colloidosome. FIG. 1C depicts colloidosome 100 with two layers of micromaterials nanomaterials, or both forming the shell. FIG. 1D depicts colloidosome 100 with two layers of micromaterials nanomaterials, or both having a space between the layers.

Referring to FIG. 1, colloidosome 100 includes a shell 102, a core 104, and active agents 106. The shell 102 can be formed of a plurality of micro- or nanomaterials 108, which are attached together to form interstices 110. Attachment of the micro- or nanomaterials can be by a covalent bond, sintering, electrostatic interaction, van der Waals interaction, ionic interaction, hydrogen bonding, dipolar interaction or any combination thereof. While micro- or nanomaterials 108 are shown in FIGS. 1A-1D as spheres, micro- or nanomaterials can have any shape or size suitable for the intended use. The cores of the colloidosome in FIGS. 1A, 1C, and 1D are shown as a void spaces. As shown in FIGS. 1A, 1C and 1D active agents 106 can be loaded in the shell nanomaterial 108, in the interstices 110 (but not in the core), on the outside surface of the shell nanomaterial 108, or any combination thereof. It should be understood that the colloidosome can have one or more active agents 106 bound to the surface of the plurality of nanomaterials, loaded or impregnated within the plurality of nanomaterials, contained within the plurality of interstices formed between the nanomaterials or any combination thereof. FIG. 1B shows micro- or nanomaterials 108 and the core 104 loaded with active agents.

Various active agent release profiles can be achieved using colloidosome of the present invention. For tuning the tenacity or flux/rate of the released agent, the micro- or nanomaterial size, number of shells, and trigger response(s) can be controlled. Smaller-sized nanomaterials and/or multiple shells or layers can produce smaller and tortuous pores in the colloidosomes. Such an architecture can have a faster response, when triggered. A constant rate of release, with or without a trigger or an increase in pore tortuosity, can be achieved by choosing larger size micro- or nanomaterials (leading to larger size pores/interstices). In some instances, the size of the micro- or nanomaterials with an increasing number of shells (layers) can facilitate tuning of the porosity and tortuosity of the overall colloidosomes architecture. In some instances, a colloidosome architecture with multiple shells of micromaterials, nanomaterials, or both (and small pore size) can be used to enable greater volume of agent-release per colloidosome particle or micro- or nanomaterial particle.

In some instances, the colloidosome can have multiple layers that make up the shell (See, FIG. 1C and FIG. 1D). The layers can contact each other (FIG. 1C). In other instances, the layers can be separated from one another and have a space there between (FIG. 1D). In either instance, multiple layers can equate to a multi-shelled structure. As shown in FIG. 1C, both layers of micro- or nanomaterials are loaded with the active agents 106 and/or the active agent is loaded in the space between the micro- or nanomaterial layers. FIG. 1D shows both layers of micro- or nanomaterials and interstices loaded with the active agents 106, and active agents between the inner shell 102′ and outer shell 102. It should be understood that the active agents can be loaded throughout, on the surface layer of micro- or nanomaterial and/or in the interstices between the micro- or nanomaterials. While agent actives 106 are shown in FIGS. 1A-1D as a single material it should be understood that shell 102 can be loaded with an effective amount of active agent or multiple active agents for the intended use(s) (e.g., 1, 2, 3, 4, 5, 6, 7, 8, 9, 10 or more active agents).

The overall size of the colloidosome can vary depending on the intended use. By way of example, the overall average size of the colloidosome 100 can be from 5 nm to 30000 nm, from 100 nm to 1000 nm, or from 200 nm to 500 nm, or greater than or substantially equal to any one of, or between any two of: 5 nm, 10 nm, 100 nm, 200 nm, 500 nm, 1000, nm 1200 nm, 1500 nm, 2000 nm, 2500 nm, 3000 nm. The average size of the plurality of nanomaterials can be 2 nm to 1500 nm, 10 nm to 1000 nm, 50 nm to 500 nm, or 100 nm to 200 nm, or greater than or substantially equal to any one of, or between any two of: or 2 nm, 5 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 150 nm, 200 nm, 250 nm, 300 nm, 350 nm, 400 nm, 450 nm, 500 nm, 550 nm, 600 nm, 700 nm, 800 nm, 900 nm, 1000 nm, 1100 nm, 1200 nm as determined by the synthesis method such as an emulsification process. The size of the colloidosome can be determined using scanning electron microscopy (SEM), transmission electron microscopy (TEM), dynamic light scattering (DLS), or colorimetric methods or UV-vis spectroscopy methods. The size of the colloidosome core can be smaller than or equal to the size of the colloidosome shell. For example, the colloidosome core size can be 5 nm to 2500 nm with the proviso that the size of the colloidosome core is larger than the average size of the plurality of nanomaterials used to make the colloidosome.

In some embodiments, a colloidosome of the present invention that includes active agents in the core can have relatively well-defined pores (interstices) whose size can be varied depending on the application. For example, if the colloidosome includes a biological cell in the core, the interstices may be sized to be large enough to allow any desirable substance produced by the biological cell to diffuse out of the core through the pores and external to the colloidosome, as well as allow desirable substances necessary to sustain the cell, such as glucose or other nutrients, to enter the core. The pores for such an application are sufficiently small or otherwise sized to prevent entry into the core of materials that could affect the cell (e.g., by immune system cells or immune system components (e.g., various antibodies)) and/or prevent the cell from exiting the core through the pores. As described herein, the pore size can be adjusted by the size of the nanomaterials utilized. Although pore size can vary depending on the application, non-limiting examples of pore sizes range from about 0.5 nm to about 3 micrometers, about 10 nm to about 1000 nm, or about 75 nm to about 200 nm, etc. In some instances, irrespective of pore size, the pore size can be tuned by surface functionality of the pore, where the surface functionalized molecule's length can act as a pore blocking or gating tether.

In certain embodiments of the invention, the interstice sizes in a colloidosome are substantially uniform. That is, at least about 90%, or about 95%, or even about 100% of the interstice of the colloidosome can be about the same size. These interstices can, for example, have the same average diameter or vary no more than about 10%, about 5%, or about 2% of the average diameter. The average diameter of a non-circular interstice is the diameter of a circle having the same surface area as that of the interstice. In other embodiments, the radius of the interstice may differ by about 50% to about 300%, resulting in interstice differing in diameter by up to a factor of about 1.5, or even by a factor up to about 4. In yet another embodiment, the interstice may differ in radius by up to about 50%.

1. Colloidosome Shell

As described above, the colloidosome shell can be defined by a plurality of micro- or nanomaterials and interstices formed between the micro- and/or nanomaterials. As shown in FIGS. 1A-1D, the colloidosome shell can be one layer or multi-layers of micro- or nanomaterials. The shell can be capable of expanding, contracting, constricting, reacting, folding, reversing its surface charge (e.g., from negative to positive) or dissolving (partially or fully) in response to a stimulus to release the active agent from the shell. By way of example, the shell can expand when contacted with water (e.g., water droplets, moisture, vapor, condensate, etc.), change its surface charge from negative to positive at a specific pH, or respond to a change in temperature or a temperature range (e.g., from 10-150° C., or 50 to 100° C.). The shell can be tuned to release the active agent and an additional (e.g., a second) active agent in response to the same or different stimuli. The micromaterials, nanomaterials, or combinations thereof in the shell can be the same or different. By way of example, a shell can have 1, 2, 3, 4, 5, 6, or more distinct micro- or nanomaterials that form a single shell.

The micro- or nanomaterials 108 can be microgel particles, nanogel particles, polymer brushes, surfactant molecules, metal oxide particles, lipid particles, block copolymers, biopolymers, biomolecules, micellar/dendrimer structures, yolk/shell structures, core/shell structures, pomegranate structures, hollow structures/micro- or nanomaterials, functionalized micro- or nanostructures, or combinations thereof that are able to self-assemble and form the colloidosome shell. The overall performance of the colloidosome and/or shell can be tuned by changing the surface of the micro- or nanomaterials. The micro- or nanomaterials can be porous to allow the active agent to flow out of the micro- or nanogel in response to the stimulus or stimuli. By way of example, the micro- or nanomaterials can be textured to facilitate surface interactions or reactions. The micro- or nanomaterial shell (e.g., layers 200 and 204) can be made from porous or hollow metal oxides, polymers, functionalized polymers, copolymers, cross-linked polymers, cross-linked functionalized polymers, cross-linked copolymers and the like. In some embodiments, the micro- or nanomaterials can include surface functional groups with a specific chain length of attached molecules, which can serve as blocking or gating agents.

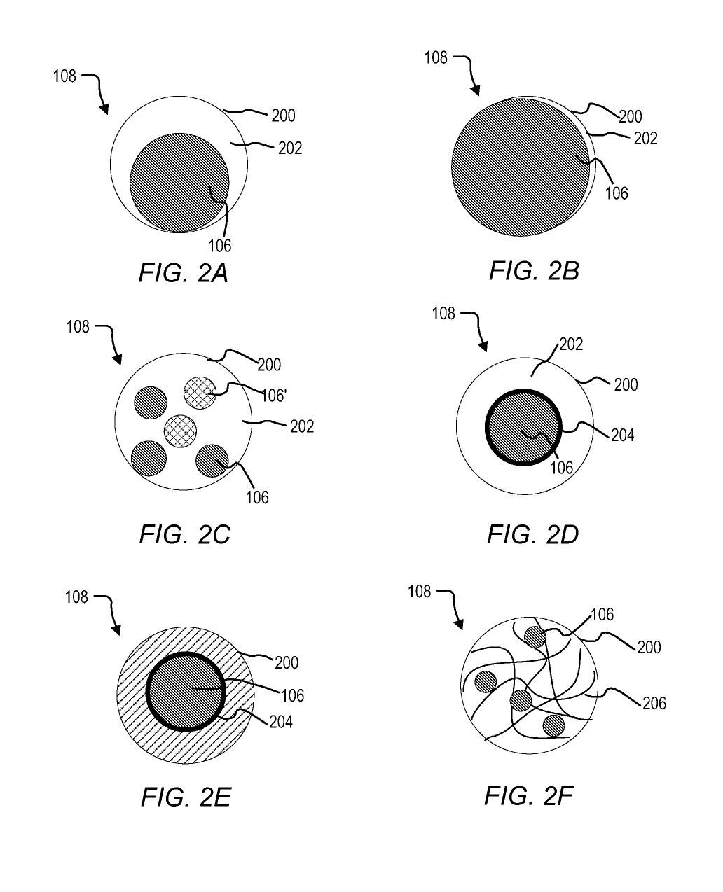

The micro- or nanomaterials can have various shapes and sizes. FIGS. 2A-2F depict various micro- or nanomaterial structures. FIGS. 2A, 2C and 2F are various types of yolk/shell micro- or nanomaterials and FIGS. 2B, 2D, and 2E core/shell type micro- or nanomaterials where active agent 106 is the yolk or core in each of the FIGS. FIGS. 2A and 2B are yolk/shell and core/shell type structures, respectively. FIG. 2C is a yolk/shell type structure, FIG. 2D is a multi-layer micro- or nanomaterial with a core, FIG. 2E is a two-layer material with a core, and FIG. 2F is a micro- or nanomaterial that includes a polymer matrix loaded with active agents. Referring to FIGS. 2A and 2B, micro- or nanomaterial 108 can include shell 200 and active agent 106 in void space 202. In some instances, void space 202 is a fluid. In FIGS. 2C and 2F, multiple yolks (active agents 106 and 106′) are shown in micro- or nanomaterials 108. FIG. 2C includes different active agents 106 and 106′ that can be released when micro- or nanomaterial 108 is subjected the same or different stimuli. FIG. 2D includes micro- or nanomaterial outer layer 200 (micro- or nanomaterial shell), micro- or nanomaterial inner layer 204, active agent 106 and micro- or nanomaterial void space or fluid 202. In FIG. 2D, active agent 106 can be encapsulated in micro- or nanomaterial inner layer 204 to form an encapsulated micro- or nanomaterial, and the encapsulated micro- or nanomaterial can then be further encapsulated in micro- or nanomaterial layer 200. Referring to FIG. 2E, active agent 106 is encapsulated in micro- or nanomaterial inner layer 204, which is then encapsulated in micro- or nanomaterial layer 200. In FIG. 2E, micro- or nanomaterial layer 200 connects to or abuts micro- or nanomaterial layer 204.

Micro- or nanomaterial layers 200 and 204 can be made of the same or different materials and can have the same or different responses to a stimulus or stimuli. For example, referring to FIGS. 2D and 2E, micro- or nanomaterial layer 200 can be made of a material that is responsive to a first stimulus and nanomaterial layer 204 can be made of a layer responsive to a second stimulus. Referring to FIG. 2F, the polymer matrix or aerogel 206 core surrounded by layer 200 can be loaded with active agents 106. In embodiments, when aerogel 206 is used, the pores can have a diameter in the range of less than 1 to 100 nanometers and preferably 1 to <20 nm, or 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm or any range or value there between. The high surface area and open-porous skeleton of the aerogel can serve as a layer around the active agents.

Micro or nanomaterial layers 200 and 204 can be made of materials that are responsive to one or more stimuli. By way of example, micro- or nanomaterial layer 200 can be made from a polymer that is sensitive to water (e.g., hydrophobic or hydrophilic) and not sensitive to pH and micro- or nanomaterial layer 204 can be responsive to pH. Upon contact with water having a desired pH, micro- or nanomaterial layer 200 can either expand (swell) or constrict (shrink) and allow the water to enter or exit the core of the micro- or nanogel. In some instances, the layer can respond to a change in humidity. The pH adjusted water can contact layer 204, and then the layer 204 can respond to the stimuli (e.g., constrict) and allow the active agent to flow out of the micro- or nanomaterial 200.

In a particular instance, micro- or nanogel particles can have a gel phase that includes a polymer network of hydrophilic, hydrophobic, amphiphilic, amphiphobic, lipophilic, lipophobic polymers, or a combination thereof. In some instances, the polymeric network can include cationic, anionic, or zwitterionic polymers or polymers comprising metal-organic frameworks (MOFs) or zeolitic imidazolate frameworks (ZIFs). The polymer network can include polyvinyl alcohol (PVA), N-isopropyl acrylamide (NIPAAm), poly(N-isopropyl acrylamide) (pNIPAAm), cross-linked poly(N-isopropylacrylamide), N,N′-methylenebisacrylamide (MBA), poly(ethylene glycol), NH₂-pNIPAAm, CO₂H-pNIPAAm, a hydroxylated poly(methylmethacrylate), an ethylene-vinyl acetate copolymer, poly(2-hydroxyethyl methacrylate) (HEMA), poly(maleic acid/octyl vinyl ether) (PMAOVE), a polyurethane, poly(acrylic acid), poly(stearyl acrylate) (PSA), poly(acrylamide) and copolymers thereof, such as dipropylene glycol acrylate caprylate (DGAC) or dipropylene glycol diacrylate sebacate (DGDS), CO₂H-pNIPAAm-MBA copolymer, NH₂-pNIPAAm-MBA copolymer, or starch, chitin or a derivative thereof, silicone or a derivative thereof, or a polyolefin, or any combination thereof.

The mechanical properties of the micro- or nanomaterial(s) are such that the colloidosome and/or shell of the colloidosome can resist premature response to a stimulus. Non-limiting examples of mechanical properties include yield strength, tensile strength, toughness and fracture toughness, stiffness, strain hardening, uniform and non-uniform elongation, creep resistance, elongation at fracture, Young's modulus, loss and storage modulus, and strain energy. Yield strength of the shell can be greater than or substantially equal to any one of, or between any two of: 0.001 kPa, 1 kPa, 5 kPa, 10 kPa, 15 kPa, 20 kPa, 25 kPa, 30 kPa, 35 kPa, 40 kPa, 45 kPa, 50 kPa, as measured by tensile testing or dynamic mechanical analyzer.

The colloidosome can be optically transparent in a medium (e.g., water). In some embodiments, the colloidosome when loaded with active agent can retain at least 50%, at least 60%, at least 70%, at least 80%, at least 90% or at least 99% transparency at concentrations less than 10%, less than 8%, less than 5% in an optically transparent medium (e.g., alcohol, water, and the like).

The micro- or nanomaterials can be made using known processes to produce a desired nanomaterial. By way of example, polymer coated silica micro- or nanoparticles can be made using sol-gel processes followed by polymer coating of the produced micro- or nanoparticles. Micro- or nanogels can be made in a manner described in the Examples. Alternatively, micro- or nanogels can be made by polymerization processes, inverse-nanoprecipitation processes, inverse micro-emulsion process, double layer emulsification process, emulsion electrospinning process, photo-crosslinking of polymers, etc. In a particular instance, a micro- or nanogel can be prepared using an inverse nanoprecipitation approach (“click” reaction) and then functionalized with a stimulus sensitive group (e.g., an ultra pH-sensitive group). Various processes to synthesize nanomaterials are described in Sharma et al., Artificial Cells, Nanomedicine, and Biotechnology, 2016; 44: 165-177, which is incorporated herein by reference. Non-limiting examples of processes to make micro- or nanomaterials include a) polymerization of monomers in a homogeneous phase or in a microscale or nanoscale heterogeneous environment, b) self-assembly of interactive polymers, c) cross-linking of pre-formed polymers, and d) emulsion photopolymerization process.

Aerogel nanomaterials can be made, for example, in an oil-in-water (O/W) emulsion with a chemical material solubilized in the silicon phases such as tetraethoxysilane (TEOS) tetramethoxysilane (TMOS), methyltrimethoxysilane (MTMS), or methyltriethoxysilane (MTES). Hydrolysis of the silicon precursor droplets and condensation of the hydrolyzed species to silica can occur at the oil-water interface and lead to formation of the hard silica shell. The conditions of the process can be controlled by various factors including acid-base catalysis, surfactants, type of silicon precursors, temperature, time, and mixing conditions. Both porosity and particle size that are critical for the release characteristics are highly tunable by the selection of process parameters and/or composition.

2. Colloidosome Core Materials

The colloidosome core can include a void space, a polymer emulsion, a polymer gel, an active agent, an inert liquid, a gas, an active agent, an aerogel, porous metal oxides, or any combination thereof. In some instances, the core is the active agent. Non-limiting examples of polymer emulsions include poly(N-isopropylacrylamide), N-isopropylacrylamide/N,N′-methylenebisacrylamide copolymer, poly(vinyl alcohol), poly(lactic acid), anionic copolymers based on methacrylic acid and methyl methacrylate (e.g., Eudragit® S100 sold by Evonik® Industries, USA), poly-e-caprolactone, poly(alkylcyanoacrylate), poly(lactide-co-glycolide), poly ethylene glycol, chitosan-based polymers. Non-limiting examples of polymer gels include poly(N-isopropylacrylamide), polyvinyl alcohol, artificial chaperone, cholesterol-bearing pullulan (CHP) nanogel, glycogen, polyethyleneglycol-b-poly(methacrylic acid), polyethylene glycol, chitosan-based materials, and alginate-based materials. Non-limiting examples of inert liquids (e.g., solvents for formulation media) include ethanol, methanol, acetone, hexane, ethylene glycol, and toluene. Non-limiting examples of gases include carbon dioxide, carbon monoxide, oxygen, hydrogen, helium, argon, nitrogen, gaseous hydrocarbons (e.g., methane, ethane, ethylene, propane, propylene, and the like), or combinations thereof. Non-limiting examples, of metal oxides include silicon dioxide, titanium dioxide, zirconium dioxides, or any combination thereof.

3. Active Agents

The active agent and/or the core active agent (an active agent positioned in the core of the colloidosome) can be a chemical agent, a biological agent, an oil, an ionic liquid, a suspension, or a polymer, or any combination thereof. Individual micro- or nanomaterial shells of the colloidosome can be loaded with multiple active agents with different triggers. Such loading can result in a multiple agent delivery system or enable rapid and local reactions (e.g., chemical) between the two released agents to result in a new product (e.g., a new chemical, biological agent or drug). The released active agents from the colloidosome (e.g., core and micro- or nanomaterials) can be different, and inhibit or repeal each other. Such delivery approaches can provide controlled and/or selective reactions. Non-limiting examples of active agents are provided throughout this specification as well as in the following subsections.

i. Chemical Agents

Chemical agents can include reactive and non-reactive agents. Reactive agents are chemicals that under a chemical reaction in the presence of another chemical or stimulus. Non-reactive chemical agents do not react in the presence of another chemical or stimulus. All types of chemical agents can be used in the context of the present invention. Non-limiting examples of chemical agents include adhesives, dyes (e.g., inks, thermochromics, etc.), cosmetic agents (e.g., cosmetic ingredients described in the CTFA International Cosmetic Ingredient Dictionary and Handbook (2004 and 2008)), pharmaceutical ingredients, pesticides, herbicides, phase-change materials, self-healing coatings, visual indicators, nanoparticles (metal or non-metal particles), imaging agents, catalysts (organic, inorganic, and organometallic), sealants, hormones, fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraaminobenzoic acid (PABA) and corresponding PABA derivatives, titanium dioxide, zinc oxide, etc.), vitamins (e.g., A, B, C, D, E, and K), trace metals (e.g., zinc, calcium and selenium), anti-irritants (e.g. steroids and nonsteroidal anti-inflammatories), antioxidants (e.g., BHT and tocopherol), chelating agents (e.g., disodium EDTA and tetrasodium EDTA), preservatives (e.g., benzoic acid, sodium benzoate, hydroxybenzoate, lactic acid, nitrite, nitrates, propionic acid, sodium propionate, sulfur dioxide, fulfities, sorbic acid, sodium sorbate, methylparaben and propylparaben), pH adjusters or buffers (e.g., sodium hydroxide, hydrochloric acid, and citric acid, and phosphates), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., glyceraol, sorbitol, urea, and manitol), exfoliants, waterproofing agents (e.g., magnesium/aluminum hydroxide stearate), and skin conditioning agents (e.g., aloe extracts, allantoin, bisabolol, ceramides, dimethicone, hyaluronic acid, and dipotassium glycyrrhizate).

ii. Pharmaceutical Agents

Non-limiting examples of pharmaceutical active agents include adjuvants, anti-acne agents, agents used to treat rosacea, analgesics, anesthetics, anorectals, antihistamines, anti-inflammatory agents including nonsteroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

iii. Nanoparticle Agents

Non-limiting examples of nanoparticles include metal particles, metal oxides, or alloys thereof, quantum dots of organic and inorganic materials, particle shaped 2D materials (small flakes) or any combination thereof. Metal particles can include alkali metals, alkaline earth metals, noble metals (e.g., gold, platinum, palladium), and transition metals (e.g., silver, chromium, copper, nickel, cobalt lanthanides and the like).

iv. Biological Agents

Biological agents include pathogens (e.g., a bacterium, a virus, a protozoan, a parasite, a fungus or prion), proteins, anti-microbial agents, DNA, microorganism, cells (e.g., a prokaryotic cell, a eukaryotic cell, a tumor cell and the like), antibodies (e.g., poly- and/or monoclonal), antibody fragments, antibody-drug conjugates, hormones (e.g., peptidic hormone, such as insulin or growth hormone, or a lipid hormone, such as a steroid hormone, for example prostaglandin and estrogen), polypeptides (e.g., a protein or a protein having catalytic activity, for example having ligase, isomerase, lyase, hydrolase, transferase or oxidoreductase activity), etc.

Non-limiting examples of viruses include adenoviridae (e.g., adenovirus), herpesviridae (e.g., Herpes simplex, type 1 and type 2, and Epstein-barr), papillomaviridae (e.g., human papillomavirus), hepadnaviridae (e.g., Hepatitis B), flaviviridae (e.g., Hepatitis C, yellow fever, dengue, West Nile), retroviridae (e.g., immunodeficiency virus (HIV)), orthomyxoviridae (e.g., Influenza), paramyxoviridae (e.g., measles, mumps), rhabdoviridae (e.g., rabies), and reoviridae (e.g., rotavirus).

Non-limiting examples of bacterium include gram-positive bacterium and a gram-negative bacterium. Non-limiting examples of gram-positive bacteria include Corynebacterium, Mycobacterium, Nocardia, Streptomyces, Staphylococcus (such as S. aureus), Streptococcus (such as S. pneumoniae), Enterococcus (such as E. faecium), Bacillus, Clostridium (such as C. difficile) and Listeria. Non-limiting examples of gram negative bacteria include Hemophilus, Klebsiella, Legionella, Pseudomonas, Escherichia (such as E. coli), Proteus, Enterobacter, Serratia, Helicobacter (such as Holicobacter pylon), and Salmonella.

v. Oils and Extracts

Oils and extracts can be classified in the following categories: (i) essential oils; (ii) aroma chemicals; (iii) absolutes; (iv) balsams; (v) concentrated oils; (vi) essences; (vii) extracts; (viii) resins; and (ix) infusions. Botanical extracts (e.g., aloe vera, chamomile, cucumber extract, Ginkgo biloba, ginseng, and rosemary) can be used as an active agent in the context of the present invention. Essential oils include oils derived from herbs, flowers, trees, and other plants. Such oils are typically present as tiny droplets between the plant's cells, and can be extracted by several methods known to those of skill in the art (e.g., steam distilled, enfleurage (i.e., extraction by using fat), maceration, solvent extraction, or mechanical pressing). Typical physical characteristics found in essential oils include boiling points that vary from about 160° C. to 240° C. and densities ranging from about 0.759 to about 1.096. Loading an oil and/or extract on a micro- or nanomaterial can inhibit evaporation of the oil and/or oxidation of the oil. Oxidation of the oil can be inhibited when the micro- or nanomaterial is made from an opaque material or a material that includes a UV blocker.

Essential oils typically are named by the plant from which the oil is found. For example, rose oil or peppermint oil is derived from rose and peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, ylang ylang, or any combination thereof. Other essential oils known to those of skill in the art are also contemplated as being useful within the context of the present invention.

Chemical compounds that impart a fragrance/odor can be used. For example, carvone, isoamyl benzoate, methyl heptine carbonate, triacetin, anethole, methyl isoeugenol, safrole, diphenyl oxide, benzyl propionate, eugenol acetate, phenylethyl acetate, cinnamyl acetate, propiophenone, p-cresyl acetate, p-methyl acetophenone, benzyl acetate, ethyl acetoacetate, ethyl benzoate, isosafrole, ethyl cinnamate, acetophenone, benzyl benzoate, p-methyoxy acetophenone, methyl cinnamate, benzyl formate, methyl benzoate, 2-undecanone, ethyl laurate, isoamyl isovalerate, 2-nonanone, linalyl acetate, octyl acetate, phenyl methyl carbonyl propionate, isoamyl butyrate, menthyl acetate, menthone, phenyl methyl carbonyl acetate, terpinyl acetate, thujone, ethyl caprylate, fenchone, geranyl acetate, bornyl acetate, pulegone, p-cresyl ethyl ether, methyl eugenol, piperitone, jasmine, methyl chavicol, dibenzyl ether or any combination thereof. Other fragrant chemical compounds known to those of skill in the art are also contemplated as being useful within the context of the present invention.

vi. Ionic liquids, Suspensions, and Polymeric Agents

Ionic liquids are ionic, salt-like materials that are liquid below 100° C. Ionic liquids can be used as solvents, separation media, electrolytes, lubricants, or the like. Ionic liquids can be miscible with water or organic solvents, thus targeted release to, or protection from, specific solvents can be accomplished with the colloidosomes of the present invention. A commercially available ionic liquid is 1-butyl-3-methylimidazolium hexafluorophosphate ([BMIM]PF₆). [BMIM]PF₆, 1-methyl-4-formylpyridinium iodide 1-hexyl-3-methylimidazolium [NTf2] ([HMIM][NTf2]) and other ionic liquids are available from various commercial vendors such as Sigma Aldrich® (USA). Non-limiting examples of other ionic liquids are described in U.S. Patent Application Publication No. 2006/0166856 to Petrat et al., which is incorporated herein by reference. Non-limiting examples of cations and anions of ionic liquids are described by Marr et al., Green Chem. 2016, 18, 105, which is incorporated herein by reference.

Non-limiting examples of a suspension includes suspensions of micro- or nanoparticles/micro- or nanostructures (e.g., aqueous suspensions of gold micro- or nanoparticles, metal oxide micro- or nanoparticles, and aerogels), drug suspensions, aqueous suspensions or solvent suspensions of the above-listed agents, buffers, and the like.

The polymer may be a synthetic polymer, a natural polymer, or a blend of both. Non-limiting examples of polymers include, a functionalized polymer, a doped polymer, polystyrene, polyacrylamide, a polypeptide or a polynucleotide, polymer coated nanoparticles/nanostructures or the like. Non-limiting examples of organic polymers include resorcinol-formaldehyde, phenol-formaldehyde, polyacrylates, polystyrenes, polyurethanes, and epoxies. Non-limiting examples of biological polymers include chitosan, alginic acid, gelatin, pectin, and agar agar.

### B. Method of Making Colloidosomes

The colloidosomes of the present invention can be made using nanoprecipitation, emulsion-diffusion, double emulsification, emulsion-coacervation, polymer-coating, layer-by-layer deposition, emulsion-evaporation, or an emulsification and flow-based processes. In a particular instance, a colloidosome of the present invention having a responsive micro- or nanostructured colloidosome shell can be formed by combining the micro- or nanomaterials and core material with a liquid medium. The liquid medium can then be mixed to obtain a plurality of micro- or nanomaterials aggregated around the core material. Alternatively, the liquid medium can be moved through a membrane to obtain a plurality of micro- or nanomaterials aggregated around the core material. The active agent can be bound to the surface of the plurality of micro- or nanomaterials, present within the liquid medium, or subsequently impregnated into or attached to the surface of the formed shell. In some embodiments, the liquid medium can be the core material. The liquid medium can be an aqueous medium or solution, an oil-in-water emulsion, or a water-in-oil emulsion.

In some instances, the micro- or nanomaterials can be dispersed in an oil (or a second phase system, water, buffer, solvent) with or without surfactant or emulsifier. The system can be agitated vigorously in a homogenizer to form micro- or nanomaterial-based colloidosome architecture. These colloidosomes can be collected by a bi-phasic separation method, centrifugation, or a filtration method. Once collected, the colloidosomes can be dispersed in a suitable solvent. In the instance when the micro- or nanomaterial has an active agent loaded therein, the re-dispersion can be done rapidly (or in continuous manner) to prevent loss of the active agent (e.g., diffusion of the active agent from a nanogel). The selection of solvent can be such that the partition coefficient of the active agent in the solvent is low. The steps of emulsification can be repeated again to obtain second, third, or more layers of micro- or nanomaterial to form a layered shell.

In other instances, a membrane can be used to force micro- or nanomaterials into the emulsification solution to result in uniformly dispersed architecture. Membrane emulsification can be done in one module with two or more membranes in series. If necessary, the size of nanomaterials in each colloidosome shell can be varied to achieve desired porosity within the shells as well as tuning the volume and release profile of each colloidosome shell. In some embodiments, the micro- or nanomaterials and the colloidosomes are made in a one-step process using a membrane.

Non-limiting examples of preparing colloidosomes are described in the Examples and throughout the specification. By way of example, a stock solution that includes a solvent with polyvinyl alcohol (PVA) or poly(N-isopropylacrylamide (pNIPAAm), pNIPAAm-MBA copolymers, cross-linked pNIPAAm, functionalized pNIPAAM, cross-linked functionalized pNIPAAm, copolymers thereof, and cross-linked copolymers thereof micro- or nanogels filled with an active agent (e.g., a fragrance such as limonene as an example of volatile molecule and eugenol/vanillin as an example of inert fragrance) can be prepared. The temperature of the stock solution can be maintained at a desired temperature (for example, between 0° C. and 100° C.). A solution of another active (e.g., another fragrance) can be added dropwise into the above stock solution. The emulsion ratio can be 1:4, 1:6, 1:8, or so on. The emulsion can be stirred for a desired amount of time (e.g., 1 to 5 hours) under agitation (e.g., at a specific rpm (100-5000 rpm)) and then sonicated for several minutes. These mixing steps can be done in a single pot vortexer or homogenizer system. If some instances, a stabilizer molecule (e.g., surfactant, emulsifier, sodium dodecyl sulfate (SDS), etc.) can be added to stabilize the emulsion. Stabilizer compounds can be purchased for commercial sources such as Dow Corning. In order to obtain a uniform size, the emulsion can be passed through a membrane module at various feeding rates ranging from μL/min to L/min to allow for a continuous production of uniformly-sized colloidosome architectures. The colloidosomes can be extracted in another medium such as water by adding the specific medium solution and enabling phase separation. The solvent media can be eliminated by washing colloidosomes with another solvent (e.g., 1,4 dioxane for the removal of isopropanol).

In some embodiments, microgels are self-assembled into colloidosomes and then bonded together using crosslinking agents in the presence of the active agent. Non-limiting examples of cross-linking agents include glutaraldehyde and 1,4-butanediol diglycidyl ether. The micro- or nanogels can be formed by mixing solutions of polymers at various rates to tune the size and/or shape of the colloidosome and/or the packing density and interstices between the packed microgels. By way of example, at slow rates (e.g. 2 mL/hr to up to 10 mL/hr) of solution mixing colloidosomes of different shapes (e.g., triangles, diamonds, spheres, etc.) can be prepared. Slow rates of mixing can also inhibit concentration of free and/or swollen microgels. In one embodiment, the colloidosomes can be prepared by obtaining a first solution that includes a polymer precursor (monomer) and a cross-linking agent. This can be mixed with a second solution that includes a cross-linking agent and optional emulsifier (e.g., a silicon polyether copolymer). The solution can be mixed (e.g., sonicated) and then heated (e.g., 30 to 100° C., 50 to 80° C., or 55 to 75° C., or about 60° C.) for a time sufficient to bond the microgels together to form the colloidosome structure. In some instances, additional cross-linking agent can be added to the solution prior to or after heating.

In some embodiments, the colloidosomes can be made in the absence of emulsifier and alcohol. In one method, an aqueous solution of microgels and active agent can be shaken at a desired rpm (e.g., 100 to 250 rpm, or 150 to 225, or 175 to 200 rpm at 25 to 30° C. for about 20 to 30 hours, and then stirred at a higher rpm (e.g., 550 to 900, 575 to 875, or 600 to 800 rpm) than the shaking rpm until an emulsion is formed (e.g., for about 20 to 30 hours). A cross-linking agent can be added, the mixture can be heated (e.g., 30 to 100° C., 50 to 80° C., or 55 to 75° C., or about 60° C.), and then aged until the reaction is deemed complete (e.g., about 0.5 hours to 100 hour, or about 10 hours to 75 hours) to form colloidosomes with a microgel shell surrounding an active agent core. In some instances, the active agent is loaded into the interstices of the shell.

In some embodiments of the present invention, the size (and thereby, the optical transparency) of the microgel particles used to form the colloidosome shell can be controlled with the addition of sodium dodecyl sulfate (SDS) during polymerization. By way of example, pNIPAAm or functionalized pNIPAAm (e.g., NH₂ or CO₂H functionalized pNIPAAm) micro- or nanostructures can be prepared (i.e., polymerized) in the presence of SDS to produce micro- or nanostructures having a diameter of 100 to 500 nm. Without wishing to be bound by theory it is believed that the SDS assists preventing aggregation of the gel particles during polymerization, resulting in smaller diameter microparticles. The packing of the micro- or nanomaterials 108 to form the colloidosome can be adjusted by tuning the surface of the micro- or nanomaterial to control the interactions between the nanomaterials 108 during assembly. The micro- or nanomaterials 108 can be attached together by a chemical bond (e.g., covalent bond), sintering, electrostatic interaction, van der Waals interaction, ionic interaction, hydrogen bonding, dipolar interaction or any combination thereof. In some instances, the polymeric micro- or nanomaterials can be heated to a temperature above the glass transition temperature of the polymeric micro- or nanomaterial. Upon heating, the polymeric micro- or nanomaterials can coalesce slightly to create bridges or “necks” between the micro- or nanomaterials to form the shell of the colloidosome. In a preferred embodiment, the colloidosomes can be crosslinked using glutaraldehyde, glyoxal, bifunctional amidoesters (e.g., dimethyl adipimidate), bifunctional epoxides (e.g., 1,4-butanediol diglycidyl ether), dimines, triamines (e.g., spermidine) or bifunctional N-hydroxysuccinimide (NHS) esters such as ethylene glycol-bis(succinic acid N-hydroxysuccinimide ester).

### C. Uses and Methods of Release

The colloidosomes of the present invention can be used in a variety of applications. By way of example, the colloidosomes can be comprised in a composition and the composition can be topically, transdermally, or orally administered to a subject. Alternatively, the composition can be applied to a surface of an inanimate object. As exemplified in the Examples section and described throughout the specification, the active agent can be released from the colloidosome when subjected to a specific stimulus. Compositions that include the colloidosomes of the present invention can include a pharmaceutical composition, a topical skin care composition, or a composition intended to be applied to an inanimate object. Non-limiting examples of uses of the colloidosomes of the present invention include fragrance release and cosmetics, drug delivery, bioanalysis, diagnostics, sensors & markers, energy storage, bio-inhibitors (repellants pesticides, herbicides), urea release, self-repair (paints, paper, textile, concrete, etc.), flame retardants, personal care (skin, hair, teeth, etc.), nutritional additives, vitamins, flavors, pigments, textile scent and care (detergents, softeners, etc.), industrial odors, animal care and the like.

In other instances, the active agent loaded colloidosomes are intended for use in chemical reactions. By way of example, upon being subjected to a proper stimulus, the colloidosome can release a first active agent, which reacts with a second active agent to form a new product. In another example, the first agent can be released and interact with a second agent to activate the second agent. In yet another example, a water-soluble polyvinyl alcohol (PVA) micro- or nanogel can swell and shrink in presence or absence of humidity. The swelling can lead to controlled release of chemicals, but at the same time, swollen PVA micro- or nanogel can close or minimize the pore size to prevent any chemicals to come out of the center core. In one particular example, a colloidosome shell of p(N-isopropyl acrylamide) (pNIPAAm) micro- or nanogels can function under temperature changes (20-100° C., preferably 25-70° C., more preferably 25 to 60° C.) to release active agents. The driving force and control of active agent release can be, in part, determined by headspace concentration. Without wishing to be bound by theory, it is believed that the controlled release in a multi-layered shell is inherited by the micro- or nanomaterial architecture due to nano/meso scale porosity (e.g., 1-20 nm) between the micro- or nanomaterial shells. This porosity can change with stimuli and time. As the largest porosity is reached, a burst release can occur. Apart from trigger mechanism and surrounding environment, permeability of the colloidosome can depend on the amount of active agents left in the core and nanogels, and the shell thickness and size of colloidosome shell.

Non-limiting examples of stimuli include pH range, electromagnetic radiation, a temperature range, a mechanical force (e.g., application or removal of pressure, a sudden change in pressure, shear force, rubbing action, and/or pulsating forces), humidity, the presence or absence of a chemical substance, an odor, or any combination thereof. A pH can be changed from acid to base or vice versa. By way of example, a pH can be changed from 1 to 12, 2 to 8, 2 to 4, 8 to 12, 12 to 5, 10 to 3, or 8 to 5. Electromagnetic radiation can include ultraviolet radiation, visible light, infrared radiation, or any combination thereof. Sources of electromagnetic radiation can include the sun and/or lamps (e.g., UV, UV/visible, visible lamps). Temperature ranges can be any range, greater than or substantially equal to any one of, or between any two of: 25° C. to 100° C., or 30° C. to 80° C., or 40° C. to 60° C., or 25° C., 30° C., 35° C., 40° C., 45° C., 50° C., 55° C., 60° C., 65° C., 70° C., 75° C., 80° C., 85° C., 90° C., 95° C., 100° C.

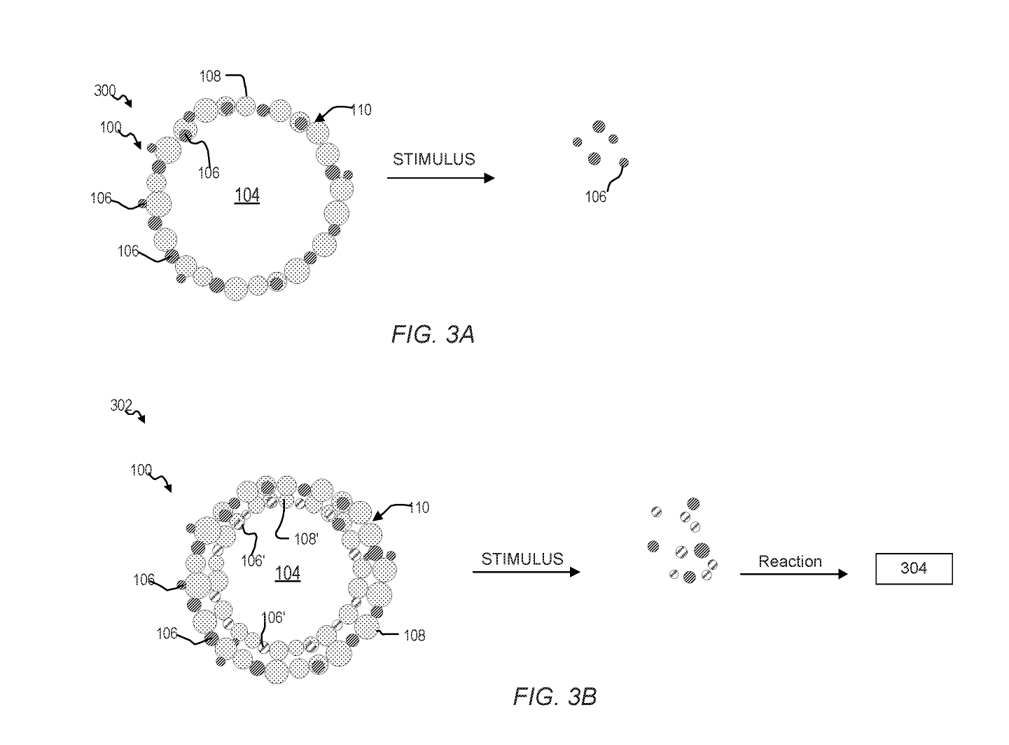

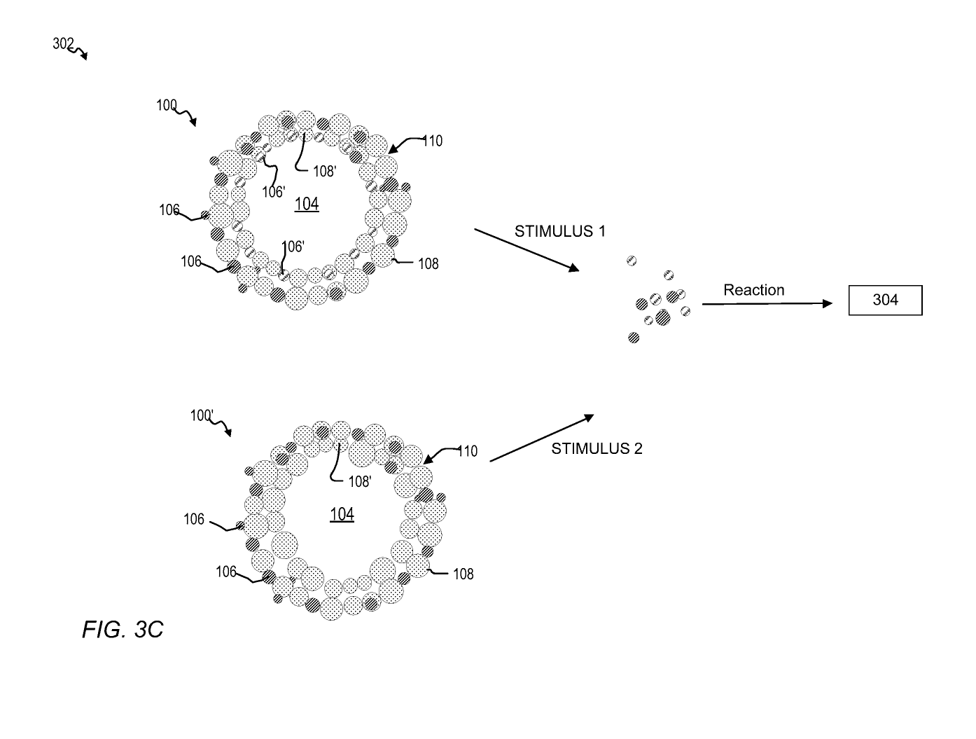

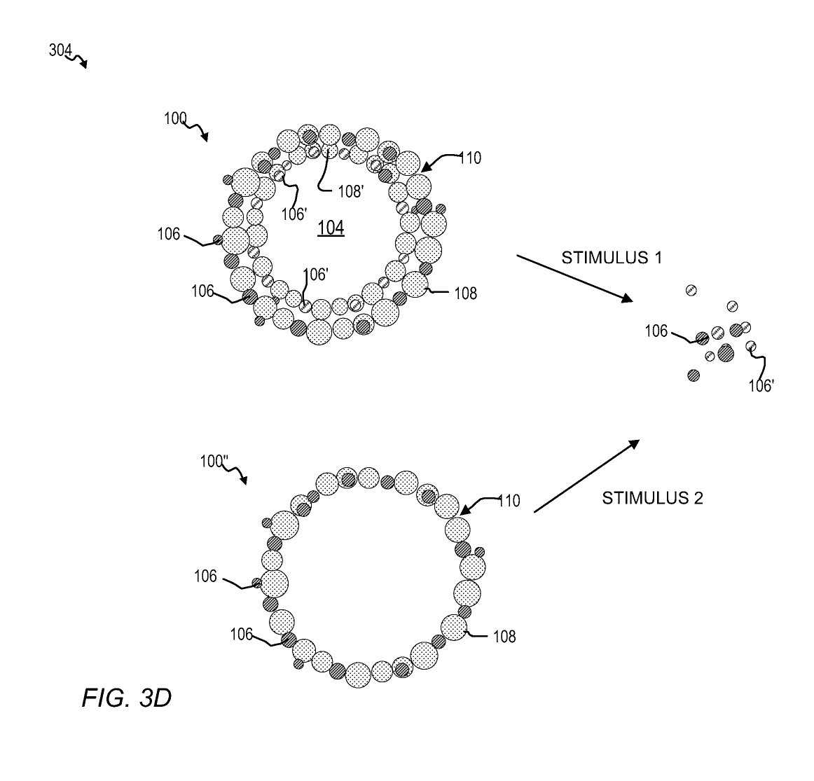

FIGS. 3A through 3D depict illustrations of method 300 and method 302 of using of the active agent loaded colloidosomes of the present invention. Referring to FIG. 3A, colloidosome 100 can be subjected to a stimulus to release active agents 106 from the micro- or nanomaterials 108.

The colloidosome of the present invention can store multiple active agents that are released in response to different stimuli. This can result in a multiple active agent delivery system or enable rapid and local active agent reaction (e.g., chemical reaction) between the two released active agents to result in a new product/chemical. The released chemicals from the core and/or shell can be the same or different. Such delivery approaches can enable controlled or selective chemical reactions leading to tunability in composition of the released product. Referring to FIG. 3B, colloidosome 100 can be subjected to a stimulus to release active agent 106 from the micro- or nanomaterials 108 located in the outer layer of the shell. Active agent 106′ present in micro- or nanomaterials 108′ can also be released in response to the same stimulus. Active agents 106 and 106′ can then react to form product 304. In some embodiments, active agents 106 and 106′ do not react to form a product 304.

By comparison, multiple stimuli can be used to release active agents 106 and 106′. In particular, and referring to FIG. 3C, colloidosome 100 loaded with active agents 106 and 106′ can be subjected to a first stimulus to release active agent 106′ from the micro- or nanomaterials 108′. This results in colloidosome 100′ (i.e., colloidosome without active agent 106′). Colloidosome 100′ can then be subjected to a second stimulus to release active agent 106 from micro- or nanomaterials 108. Active agents 106 and 106′ can then react to form reaction product 304. In some embodiments, active agents 106 and 106′ do not react to form a product 304.

In some embodiments, the triggered release of the active agents from the micro- or nanomaterials can be an initial burst release of active agent (e.g., from the core). This can be followed by controlled and sustained release of the same or different active agents from the shell. In other instances, a reverse function could be achieved where after a controlled or prolonged release of active agents in the outer shell, a burst release can occur from the core and/or optionally an inner shell if present. The latter release can indicate the end of release profile. Referring to FIG. 3D, colloidosome 100 in method 304 can be subjected to a stimulus such that micro- or nanomaterials 108′ burst to release agents 106′, thereby resulting in the formation of colloidosome 100″. Active agents 106 can then be released from micro- or nanomaterials 108, when subjected to a second stimulus.

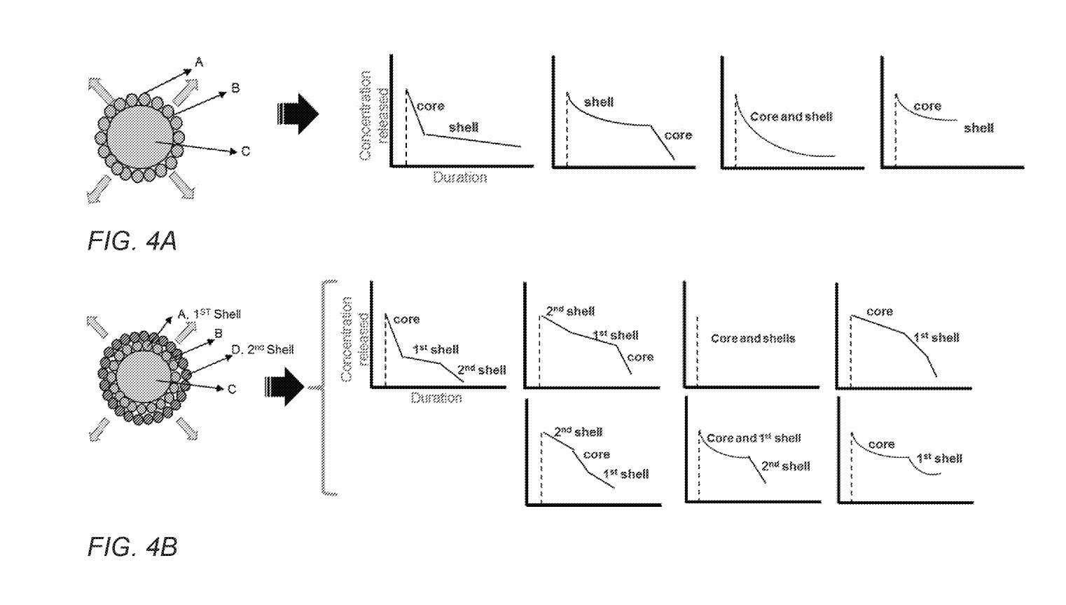

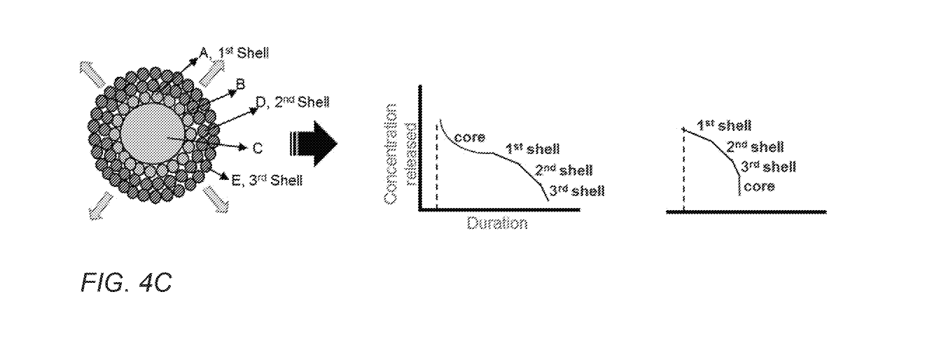

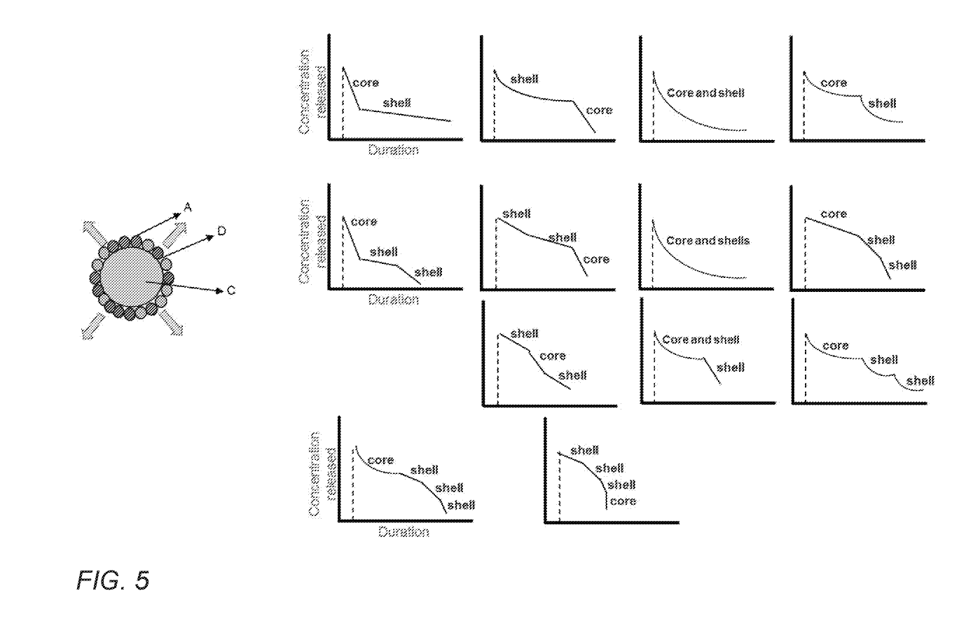

The colloidosomes of the present invention can be designed to have various release profiles. By way of example, FIGS. 4A-4C illustrate release profiles of a colloidosome having the micro- or nanomaterial, interstices B, and core loaded with active agents A, C, D, and E. FIG. 5 illustrates potential release profiles of a single shell colloidosome loaded with multiple active agents A, C, and D. Referring to FIG. 4A, it illustrates possible release profiles of a colloidosome having the micro- or nanomaterial, interstices, and core loaded with active agents A and C, respectively. Depending on the micro- or nanomaterial used and the stimulus or multiple stimuli, the release profile can be: 1) active agent C is released first from the core and then active agents A is released from the shell (two profiles are illustrated); 2) active agents A is released from the shell and then active agent C is released from the core; or 3) simultaneous or substantially simultaneous release of active agent A from the shell and active agent C from the core. FIG. 4B illustrates possible release profiles of a colloidosome having a first micro- or nanomaterial shell, interstices B between the micro- or nanomaterial, a core, and a second outer colloidosome shell loaded with active agents A, C, and D, respectively. Depending on the micro- or nanomaterial used and the stimulus or multiple stimuli, the release profile can be: 1) active agent C is released first from the core, then sequential release of an active agent from the first shell and second shell (e.g., active agents A and D, or active agent D), respectively; 2) active agent D is released from the second colloidosome shell, one and/or two active agents are released from the first colloidosome shell, and then active agent C is released from the core; 3) simultaneous or substantially simultaneous release of active agent C from the core and active agent A from the first shell and one agent from the second shell; 4) active agent D is released from the second colloidosome shell, active agent C is released from the core, and then one and/or active agent A is released from the first colloidosome shell; or 5) simultaneous or substantially simultaneous release of one active agent A from the colloidosome first shell and active agent C from the core, and then active agent D is released from the second colloidosome shell. FIG. 4C illustrates possible release profiles of a colloidosome having the first micro- or nanomaterial shell, interstices B between the micro- or nanomaterials, a core, a second (middle) colloidosome shell, and a third colloidosome (outer shell) loaded with active agents A, C, D, and E respectively. Depending on the micro- or nanomaterial used and the stimulus or multiple stimuli, the release profile can be: 1) active agent C is released first from the core, release of active agent A from the first colloidosome shell, active agent D is released from the second colloidosome shell, and active agent E is released from the third colloidosome shell; or 2) active agent A is released from the first colloidosome shell, active agent D is released from the second colloidosome shell, active agent E is released from the third colloidosome shell, and then active agent C is released first from the core.

FIG. 5 illustrates release profiles of a single shell colloidosome. The shell includes of different micro- or nanomaterials that can be loaded with the same or different active agents (e.g., A and D). The core can be loaded with active agent C. Depending on the micro- or nanomaterial used and the stimulus or multiple stimuli, the release profile can be: 1) release of active agent C from the core followed by release of one active agent A and/or D; 2) release of one active agent A and/or D from the shell followed by release of active agent C from the core; 3) simultaneous release of all active agents A, C, and D from the colloidosome; 4) release of active agent C from the core, followed by release of active agent A from the shell, followed by release of active agent D from the shell; 5) sequential release of two active agents A and D from the shell followed by release of active agent C from the core; 6) simultaneous release of two active agents A and D from the shell and the active agent C from the core; 7) release of active agent A from the shell, followed by release of active agent C from the core, following by release of active agent D from the shell; 8) simultaneous release of active agent A from the shell and active agent C from the core followed by release of active agent D from the shell; 9) release of active agent C from core followed by sequential release of active agents A, D and E (not shown) from the shell; or 10) sequential release of active agents A, D, and E (not shown) from the shell followed by release of active agent C from the core.

### EXAMPLES

The present invention will be described in detail by way of specific examples. The following examples are offered for illustrative purposes only, and are not intended to limit the invention in any manner. Those of skill in the art will readily recognize a variety of noncritical parameters, which can be changed or modified to yield essentially the same results.

### Examples 1A-1D

### Microgel and Functionalized Microgel Synthesis

### Example 1A: pNIPAAm Microgel Synthesis

Microgels of poly(N-isopropylacrylamide) were prepared using the procedure of Acciaro et al., Langmuir, 2011, 27, 7917. Approximately NIPAAm monomer (1 g, Acros Organics™ by ThermoFisher Scientific, U.S.A) was dissolved in deionized (DI) water (70 mL). The resulting mixture was vacuum filtered through 1 μm filter paper (Whatman™) which was then transferred to a reaction vessel (3-neck flask) equipped with a condenser and purged with inert gas (N₂). The solution was mixed with 0.16 gm of MBA (Fisher Chemicals, U.S.A.) under agitation, and heated to about 70° C. under N₂ for 30 min. Potassium persulfate solution (K₂S₂O₈, 0.042 g, (Acros) in 5 ml of water) was added to the solution and the reaction was stirred at about 70° C. for 1 hr, then removed from the heat bath and rapidly cooled by addition of room temperature deionized water (20 mL). The product suspension was transferred to dialysis tubing (Thermo, Snakeskin MWCO 10 kDa) that had been presoaked in water. The tubing was slowly stirred in 1.5 L of water for 5 d with 4 water changes.

### Example 1B: NH₂-pNIPAAm Microgel+SDS Synthesis

NIPAAm monomer (1 g), N,N′-methylene bisacrylamide (0.061 g, Fisher Chemicals), allylamine (0.066 mL, Alfa, U.S.A., approximately 9:1 NIPAAm:allylamine molar ratio), and sodium dodecylsulfate (0.125 g, Sigma-Aldrich®, U.S.A.) were dissolved in DI water (70 mL) that had be previously degassed by purging with nitrogen through a gas dispersion tube for >0.25 h. The solution was stirred and heated to about 70° C. under N₂ for 30 min. Potassium persulfate solution (K₂S₂O₈, 0.039 g (Acros) in 5 ml of water) was added dropwise to the solution and the reaction was stirred at about 70° C. for 1 hr, then removed from the heat bath and rapidly cooled by addition of room temperature deionized water (20 mL). The product suspension was transferred to dialysis tubing (Thermo, Snakeskin MWCO 10 kDa) that had been presoaked in water. The tubing was slowly stirred in 1.5 L of water for 5 d with 4 water changes to form NH₂-functionalized p-NIPAAm microgels.

### Example 1C: NH₂-pNIPAAm Microgel—SDS Synthesis

NIPAAm monomer (8.1 g), N,N′-methylene bisacrylamide (0.32 g), and allylamine (0.56 mL, approximately 9:1 NIPAAm:allylamine molar ratio) were dissolved in DI water (0.3 L) that had be previously degassed by purging with nitrogen through a gas dispersion tube for >0.25 h. The solution was stirred and heated to about 70° C. under N₂ for 30 min. Potassium persulfate solution (K₂S₂O₈, 0.4 g (Acros) in 5 ml of water) was added dropwise to the solution and the reaction was stirred at about 70° C. for 2.5 hr, then removed from the heat bath and filtered through a 100 micron filter cloth and placed on ice. The product suspension was transferred to dialysis tubing (Thermo, Snakeskin MWCO 10 kDa) that had been presoaked in water. The tubing was slowly stirred in 7 L of water for 5 d with 4 water changes. A portion of the microgel suspension was transferred to a polypropylene bottle, frozen on solid carbon dioxide and lyophilized to produce an off-white solid foam after 3 days. The lyophilized microgels could be resuspended in deionized water with vigorous stirring prior to use.

### Example 1D: CO₂H-pNIPAAm Microgel Synthesis

NIPAAm monomer (2.5 g), N,N′-methylene bisacrylamide (0.0.18 g, Fisher Chemicals), acrylic acid (0.130 mL, Alfa, U.S.A., approximately 9:1 NIPAAm:acrylic acid molar ratio), and sodium dodecylsulfate (0.32 g, Sigma-Aldrich®, U.S.A.) was dissolved in DI water (0.1 L) that had be previously degassed by purging with nitrogen through a gas dispersion tube for >0.25 h. The solution was stirred and heated to about 68° C. under N₂ for 30 min. Potassium persulfate solution (K₂S₂O₈, 0.11 g (Acros) in 5 ml of water) was added dropwise to the solution and the reaction was stirred at about 68° C. for 3 hr, then removed from the heat bath and rabidly cooled by addition of room temperature deionized water (20 mL). The product suspension was transferred to dialysis tubing (Thermo, Snakeskin MWCO 10 kDa) that had been presoaked in water. The tubing was slowly stirred in 1.5 L of water for 5 d with 4 water changes to produce CO₂H-functionalized pNIPAAm.

### Example 2A

### General Continuous Addition Method of Making a pNIPAAm/Limonene Colloidosome

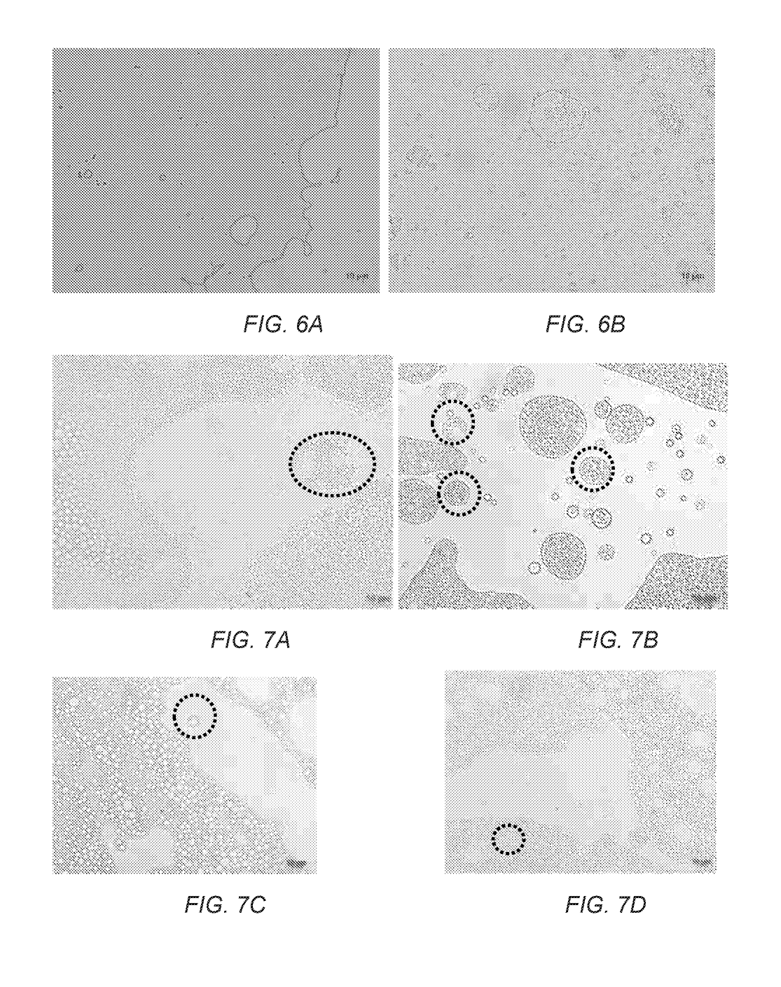

A first solution (Solution A) NH₂-pNIPAAm microgel suspension (1.73 mL, prepared by Example 1C), ethanol (15.6 mL), water (2.6 mL) and glutaraldehyde (25 wt. %, 1 to 6 mL, Fisher Scientific by ThermoFisher Scientific, U.S.A) was prepared. The total volume of Solution A was about 20 mL. A second solution (Solution B) of limonene (5 g, Acros Organics™) and formulation aid, DC5200, (1 g, Dow Chemical, U.S.A.) was prepared. The total volume of solution B was less than 10 mL. The solutions were each loaded into individual syringes. The syringes were affixed to a syringe pump and connected to a common line via a T-connector. The solution were injected at a constant rate (2 mL/hr, 10 mL/hr or 60 ml/hr), which allowed Solutions A and B to mix in-situ in an emulsified system. The emulsified system was treated at 60° C. for 30 min in a heated chamber to form swollen microgels. Subsequently, the emulsified solution of microgels was aged for more than 72 hours to cross-link the microgels and form the colloidosomes structure. The colloidosome having a plurality of micro- or nanomaterials and interstices formed between the micro- or nanomaterials, where the core of the shell is loaded with the limonene (active agent) that is capable of being released from the shell in response to a stimulus. Optical images of microgels and colloidosomes of the present invention were taken using a Carl Zeiss Microscope (Carl Zeiss, LLC, U.S.A.) with an Axio imager M2m. FIGS. 6A-B show the optical images of swollen microgels prior to aging (FIG. 6A) and after aging (FIG. 6B).

### Examples 2B Through 2D

### Continuous Addition Method of Making a polyNIPAAm/Limonene Colloidosome

### Example 2B

The procedure of Example 2A was repeated with the following changes. A premixed suspension of NH₂-pNIPAAm microgel solution (Example 1C, about 73 mL) and glutaraldehyde (25 wt. % in water, 6 mL) was used. The premixed suspension enabled site-specific cross-linking, where glutaraldehyde was adsorbed on the microgels. At 60 mL/hr about 19.5 wt. % of the limonene was loaded on the colloidosome as determined using Thermogravimetric (TGA) analysis. Samples for TGA analysis using a TA Instruments Discovery System (U.S.A.) were collected by washing and centrifuging at 1000 rpm for 10 minutes. The limonene loading was defined by wt. of limonene divided by weight of total sample (wt. of oil+wt. of polymer).

### Example 2C

The procedure of Example 2A was repeated with the following changes. The resulting emulsion was heated up to 120° C. for 1 hr. The shrunken microgels were shaken and stirred for 2 hours and left at room temperature to swell. This enabled loading or encapsulation of oil by squeezing/swelling action.

### Example 2D

The procedure of Example 2A was repeated with the following changes. A rate of 2 mL/hr was used for the re-addition of glutaraldehyde. The resulting emulsion from the continuous addition method was taken (3-10 ml of emulsion) and 3-6 ml of glutaraldehyde was added. The suspension was shaken for 2 hours and then heated at 60° C. for 30 min. Finally, the suspension was aged for more than 72 hours while shaking at 200 rpm. This method resulted in greater extent of cross-cross-linking and allowed for forming colloidosomes such as multi-shell, nested/yolk shell, and irregular architectures.

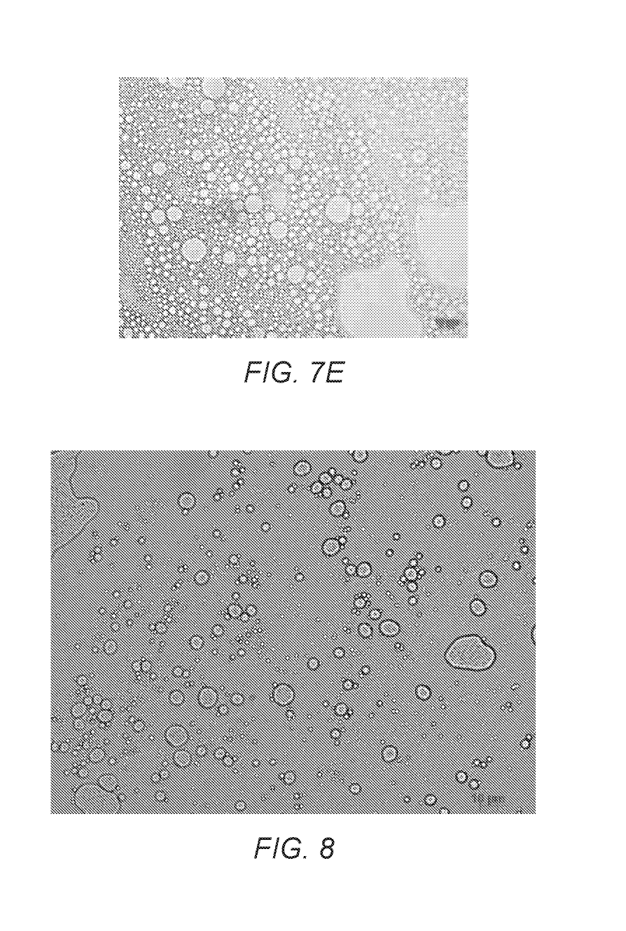

FIGS. 7A-E show images of the colloidosome structures. FIG. 7A is an optical image that shows a multi-shell (>3) colloidosome structure. The colloidosome is in the dotted line. FIG. 7B shows optical images of a nested yolk-shell structure. The colloidosome are in the dotted line. FIG. 7C shows an optical image colloidosome with a wedge-shape interstices. The colloidosome is in the dotted line. FIG. 7D shows an optical image of multi-shell (2-3) colloidosome structure. The colloidosome is in the dotted line. FIG. 7E shows optical images a multitude of single shell colloidosome structures. In each of the colloidosomes shown in the dotted circle(s) and in FIG. 7E, the limonene is surrounded by the polymeric micromaterial.

### Example 3

### General Method of Making NIPAAm or Functionalized NIPAAm/Limonene Colloidosomes

NIPAAM microgels from Example 1B (23 g suspension in water, 4 mg solids/mL and approx. 0.1-0.5 μm diameter), glutaraldehyde (25 wt. %, 0.5 mL) were prepared as Solution A. Limonene (1.25 g) and DC5200 emulsifier (0.14 g) were prepared as in Example 2A as Solution B. The two solutions were contacted to form a pNIPAAm microparticle shell surrounding the limonene core. The solution was probe sonicated, and then heated at 70° C. for about 0.5 h, then aged for 72 h.

### Example 4A

### (Method of Making Amino-NIPAAm/Limonene Colloidosomes with Ethylene glycol-bis(succinic acid N-hydroxysuccinimide ester) Cross-Linking Agent

Reconstituted lyophilized NIPAAm microgels from Example 1C (50 mg, approx. 0.5 to 1.0 μm diameter), and water (5 g) were prepared. Limonene (0.6 g) was added with vigorous stirring. 1,4-Butanediol diglycidyl ether (84 mg, 20× reactive equivalents, CAS No. 2425-79-8) was added in portions. The solution was aged at room temperature with stirring to form colloidosomes with a cross-linked pNIPAAm polymer shell surrounding the limonene core.

### Example 4B

### Method of Making Amino-NIPAAm/Limonene Colloidosomes with 1,4-Butanediol Diglycidyl Ether Cross-Linking Agent

Reconstituted lyophilized NIPAAM microgels from Example 1C (50 mg, approx. 0.5 to 1.0 μm diameter), and water (5 g) were prepared. Limonene (0.6 g) was as added with vigorous stirring. Ethylene glycol-bis(succinic acid N-hydroxysuccinimide ester) (49 mg, 5× reactive equivalents, CAS No. 70539-42-3) was added in portions. The solution was aged at room temperature for 2 d with stirring to form pNIPAAm polymer shell surrounding the limonene core.

### Example 5

### Colloidosomes from Multiple Sizes of Microgels-Shaking Method

A portion of the reaction product from Example 4A (1 g, synthesized with approx. 0.5 to 1.0 μm diameter NH₂-pNIPAM microgels) was mixed with a suspension of NH₂-pNIPAM microgels (3 g, 5 mg pNIPAM/g suspension synthesized according to Example 1B, approx. 0.2 to 0.5 μm diameter microgels), and shaken on a rotary orbital shaker. Additional 1,4-butanediol diglycidyl ether (30 microliters) was added and the reaction was shaken at room temperature overnight to form colloidosomes having multiple sizes of a microgels forming a shell and a limonene core. FIG. 8 is an optical image of the mixture of colloidosomes made of various sizes of microgels.

### Example 6

### General Centrifugation Method of Making a pNIPAAm/Limonene Colloidosome

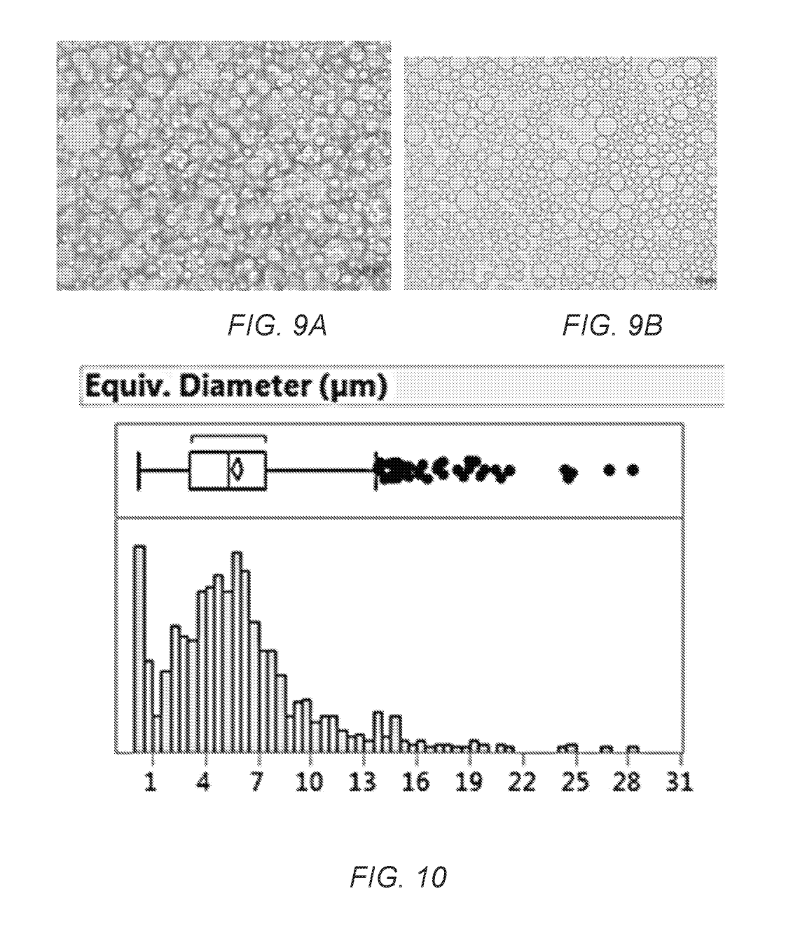

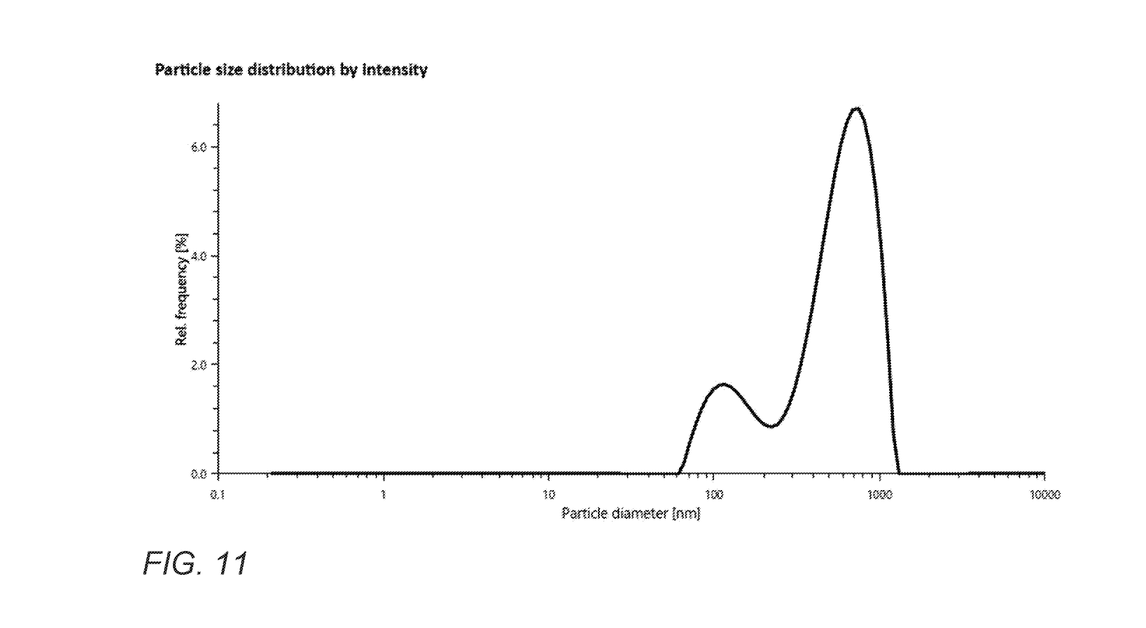

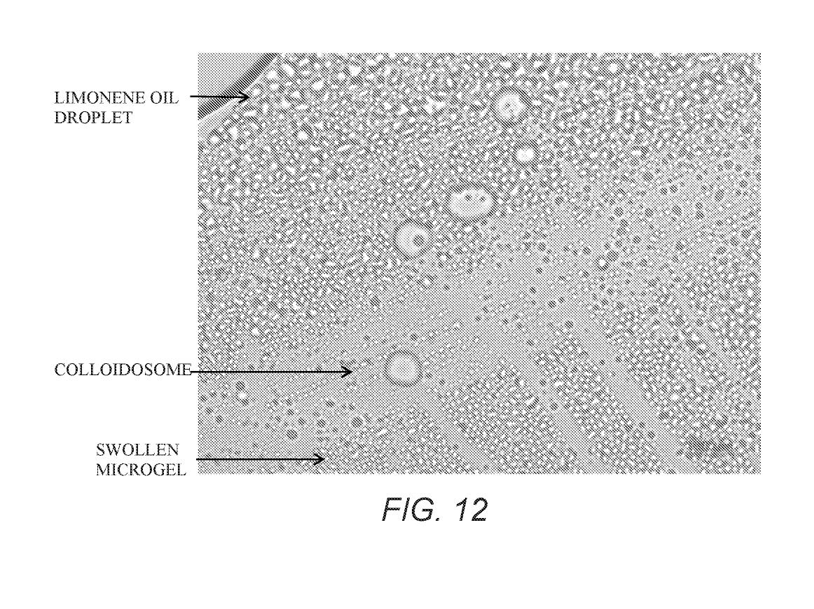

A suspension of NH₂-functionalized pNIPAAm microgels of NIPAAm in water (approx. 10 mg pNIPAAM/g suspension synthesized according to Example 1C, 30 to 50 mL solution) was centrifuged and the solids were collected. Subsequently, limonene (1 mL) was added to the collected wet microgel solids. The mixture was shaken at 200 rpm for 24 h and then stirred an additional 24 h at 600 to 800 rpm. A cream-like emulsion formed. The emulsion was mixed with 3 to 6 mL of glutaraldehyde (25 wt. % in water) and heated at 60° C. for 30 minutes. The resulting mixture was aged for more than 72 hours to form a cream-like emulsion with colloidosome structures. FIG. 9A shows an optical image of swollen NH₂-functionalized NIPAAm microgels and FIG. 9B shows an optical image of cross-linked microgels having limonene encapsulated within the colloidosome. FIG. 10 is the average diameter size distribution data for the colloidosome of FIG. 9B obtained using an Anton Paar Litesizer™ (Anton Paar, GmBH, Germany). The mean diameter was about 5.8 microns (5800 nm). Table 2 lists the quantiles data. FIG. 11 shows the relationship of particle-diameter (nm) to distribution by intensity for microgels (peak at about 100 nm) and colloidosomes (peak at about 915).

TABLE 2
Quantiles

| | | |
|---|---|---|
| 100% | 28.30 | Maximum |
| 99.5% | 24.70 | |
| 97.5% | 17.00 | |
| 90.0% | 11.10 | |
| 75.0% | 7.40 | quartile |
| 50% | 5.27 | median |
| 25% | 3.14 | quartile |
| 10% | 0.72 | |
| 2.5% | 0.23 | |
| 0.5% | 0.23 | |
| 0.0% | 0.23 | |

### Example 7

### General Sonication Method of Making a polyNIPAAm/Limonene Colloidosome

Microgels of NH₂-NIPAAm in water (10 mL, synthesized according to Example 1C) were mixed with ethanol (90 to 99.5 mL and water (5 mL to 25 mL). To the above solution, glutaraldehyde (25 wt. % in water, 2 ml to 6 ml) was further added and mixed. Another solution mixture was prepared by mixing limonene (0.1 gm to 10 gm) and emulsifier (DC5200 (Dow Corning, (U.S.A.), 0.001 gm to 1 gm). The two solution mixtures were mixed together and then sonicated (in a sonic dismembrator) for a specific duration (1 min to 10 min) at the power amplitude of 50%. The sonicated solution turned from colorless/cloudy solution to homogeneous milky solution. This solution was treated at 60° C. for 30 min. in a heated chamber. Subsequently, the emulsified solution was aged for more than 72 hours to form a cream-like emulsion with colloidosome structures. The yield of these colloidosomes was less than the yield realized in Examples 2A-2D. FIG. 12 shows an optical image of a mixture of colloidosomes of the present invention, swollen microgels and free limonene droplets. About 8 wt. % of the limonene was loaded on the colloidosome as determined by TGA analysis.

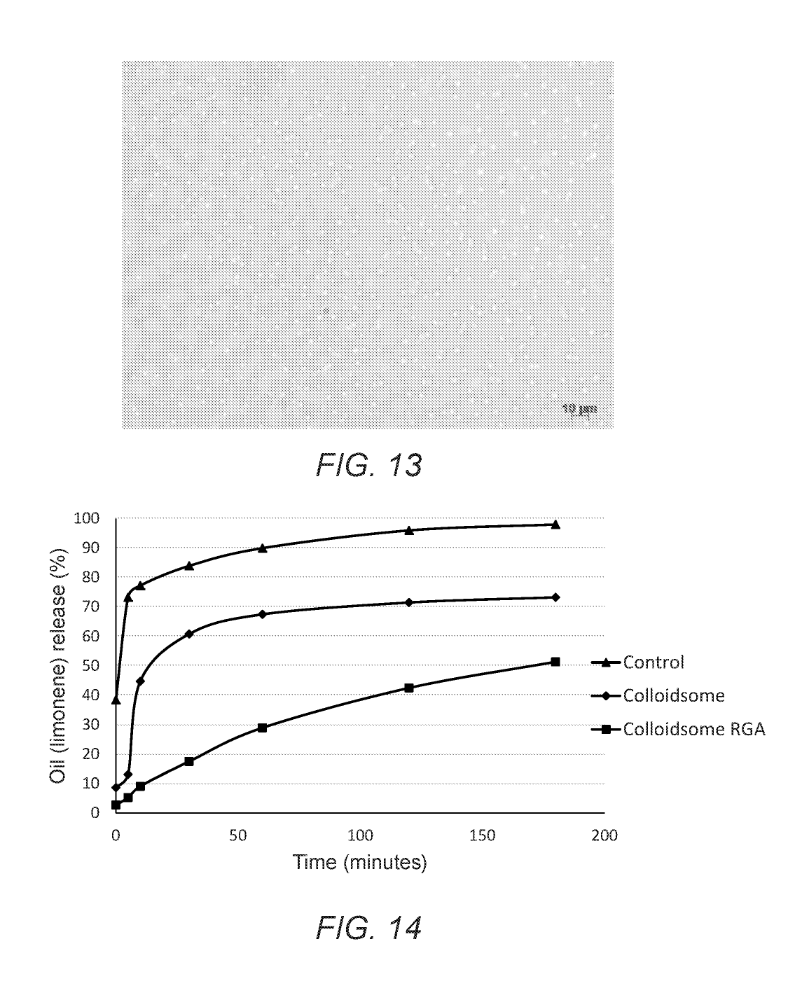

The methods demonstrated in Examples 2A-2D show tunability of colloidosome sizes as a function of addition rate and/or preparation method. By way of example, Examples 2A-2D can be used to prepare (e.g., at the slowest rate 2 mL/hr) a higher colloidosome yield than sonication. In yet another example, centrifugation can be used for high loading (e.g., 50 to 70 wt. %) of active agent. In still another example, different colloidosome shapes can be formed (e.g., triangles, circles, diamonds, rectangles) were observed at the 2 mL/hr rate. FIG. 13 shows colloidosomes with different shapes.

### Example 8

### Release of Limonene from Colloidosomes

The release of active agent from the colloidosomes of the present invention was determined. Colloidosomes from Example 2A (50 μL) or Example 2D were taken into a dialysis cassette (ThermoFisher Scientific (U.S.A.) Slide-A-Lyzer; 2,000 MWCO, 3 mL capacity with ethanol (1.5 mL). The cassette was transferred to a polyethylene bag containing ethanol (20 mL). The amount of limonene that diffused through the membrane of dialysis cassette into the ethanol solution in the bag at 23° C. was measured by absorbance. Absorbance measurements of limonene in the ethanol outside the cassette were recorded using UV-Vis-Near infrared spectrophotometer (Ocean Optics (U.S.A.)) at a wavelength of 235 nm, 1 cm path length cuvette until equilibrium was reached. FIG. 14 shows the relationship of limonene concentration over time of the colloidosomes of the present invention and a control. The control was 25 μL of limonene placed in the dialysis cassette. From the data, it was determined that addition of cross-linker in Example 2D produced a slower release of limonene as compared to the control and the colloidosome delivery system of Example 2A.

### Example 9

### Temperature Effect on the Release of Limonene from Colloidosomes

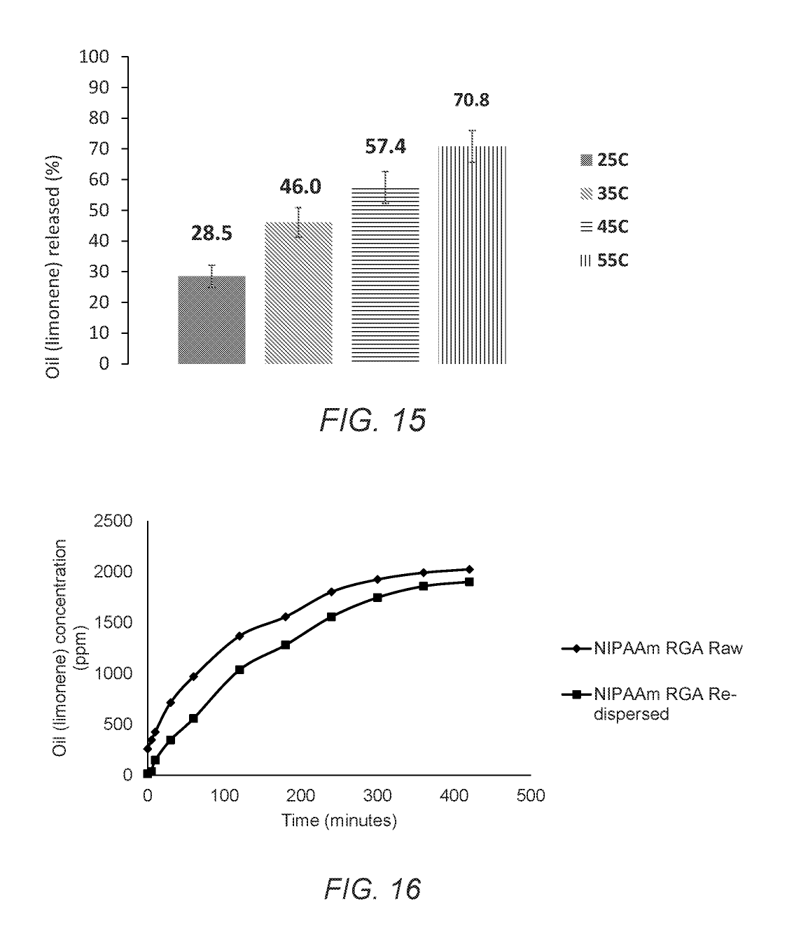

The effect of temperature on the release of limonene from the colloidosomes of the present invention was determined on four samples of colloidosomes made using the method of example 2D using the same method described in Example 8 except the bag containing the cassette was heated to 25° C., 35° C., 45° C., or 55° C. FIG. 15 shows the relationship of released limonene from colloidosomes of the present invention versus temperature over 1 minute. From the data, it was determined that the release of limonene from the colloidosome delivery system of the present invention could be controlled and/or triggered by heating the colloidosomes to a desired temperature. Table 1 lists the amounts of limonene released.

TABLE 1

| | Temperature | % Limonene |
|---|---|---|
| Sample No. | ° C. | released |
| 1 (Example 2D) | 25 | 28.5 |
| 2 (Example 2D) | 35 | 46.0 |
| 3 (Example 2D) | 45 | 57.4 |
| 4 (Example 2D) | 55 | 70.8 |

### Example 10

### Release of Active Agent from Colloidosomes in a Composition

The release of active agent from the colloidosomes of the present invention when comprised in a composition was determined. Composition samples were prepared by adding limonene loaded colloidosomes (0.5 g) from Example 2D to 20% water in ethanol solution (0.5 g). The amount of limonene released was determined following Example 8. FIG. 16 shows the amount of limonene released over time for colloidosomes of the present invention in water (top line) and in a composition (bottom line).

### Example 11

### Optical Transparency

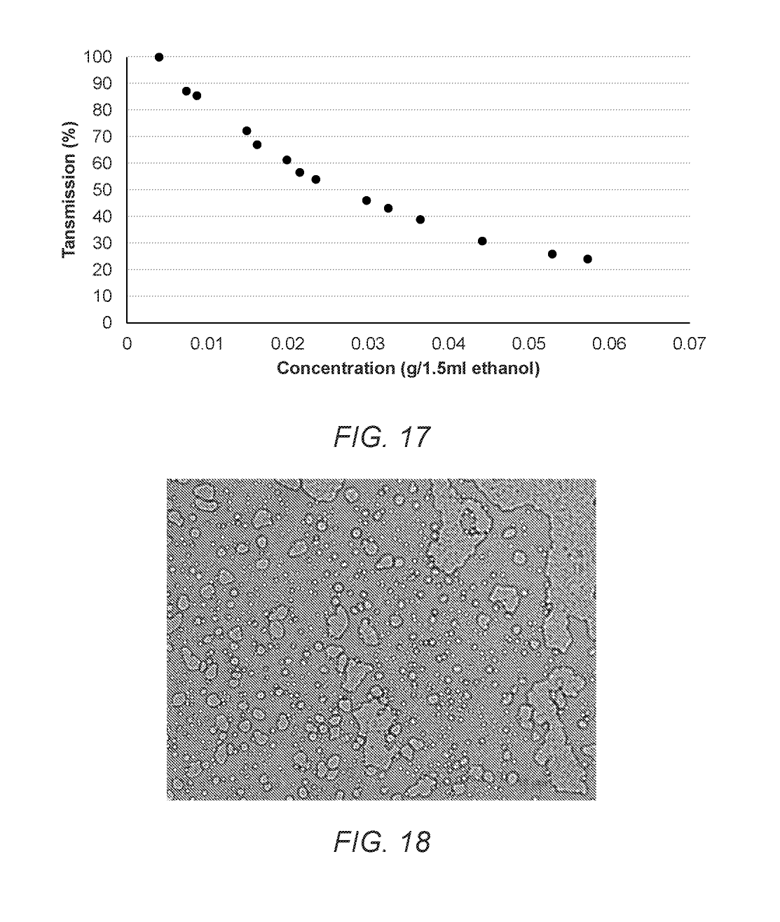

The optical transparency of the loaded colloidosomes of the present invention at various concentrations was determined. Samples were prepared by adding known amounts of pNIPAAm colloidosomes (from 0.005 to 0.059 g) from Example 2D to ethanol (1.5 mL), and measuring the transmission of the sample using an Ocean Optics UV-Vis-NIR spectrometer. FIG. 17 shows the relationship between percent transmission and concentration of colloidosomes of the present invention. From the data, it was determined that the colloidosomes loaded with active agent (limonene) retained greater than 50% transparency at concentrations less than 5%.

### Example 12

### Comparative Sample—Colloidosomes Prepared without Cross-Linking Agent

Colloidosomes were prepared as described in Example 4 for NH₂-pNIPAAm was followed except that no cross-linker was used. Upon dilution in ethanol, an even distribution of microgels instead of colloidosomes was observed by optical microscopy, consistent with no cross-linker holding the structure together. FIG. 18 shows the optical image of the microgels emulsion in water.

1. A colloidosome comprising:
(a) a responsive micro- or nanostructured porous shell defined by a plurality of micro- or nanomaterials and interstices formed between the micro- or nanomaterials, wherein the shell is loaded with an active agent that is capable of being released from the shell in response to a stimulus; and
(b) a core that is defined by the responsive micro- or nanostructured porous shell.

2. The colloidosome of claim 1, wherein the plurality of micro- or nanomaterials are microgel particles, nanogel particles, polymer brushes, surfactant molecules, metal oxide particles, lipid particles, block copolymers, cross-linked polymers, biopolymers, biomolecules, micellular/dendrimer structures, yolk/shell structures, core/shell structures, pomegranate structures, hollow structures/nanomaterials, functionalized micro- or nanostructures, or combinations thereof.

3. The colloidosome of claim 2, wherein the plurality of nanomaterials are micro- or nanogel particles having a gel phase comprising a polymer network of hydrophilic, hydrophobic, amphiphilic, amphiphobic, lipophilic or lipophobic polymers, or a combination thereof.

4. The colloidosome of claim 3, wherein the polymer network comprises a crosslinked polymer or co-polymer and wherein the polymer comprise poly(N-isopropylacrylamide) (pNIPAAm), pNIPAAm-poly(N,N′-methylenebisacrylamide) copolymer, poly(ethylene glycol), functionalized (pNIPAAm), polyvinyl alcohol (PVA), a hydroxylated poly(meth)acrylate, an ethylene-vinyl acetate copolymer, 2-hydroxyethyl methacrylate (HEMA), poly (maleic acid/octyl vinyl ether) (PMAOVE), a polyurethane, poly(acrylic acid), poly(stearyl acrylate) (PSA), poly(acrylamide) and copolymers thereof; or a polyolefin, or any combination thereof.

5. The colloidosome of claim 1, wherein the stimulus is a temperature range, a pH range, electromagnetic radiation, a mechanical force, humidity, the presence or absence of a chemical substance, an odor, or any combination thereof.

6. The colloidosome of claim 1, wherein the shell has a yield strength of 1 kPa to 1 MPa or 1 kPa to 50 kPa.

7. The colloidosome of claim 1, wherein the active agent is bound to the surface of the plurality of micro- or nanomaterials, loaded or impregnated within the plurality of micro- or nanomaterials, or contained within the plurality of interstices formed between the micro- or nanomaterials.

8. The colloidosome of claim 7, further comprising a second active agent.

9. The colloidosome of claim 8, wherein the active agent and the second active agent are different and optionally are capable of reacting with one another upon their release from the shell to form an activated material.

10. The colloidosome of claim 8, wherein the shell is capable of releasing the active agent and the second active agent in response to the same or different stimuli.

11. The colloidosome of claim 1, further comprising a second responsive micro- or nanostructured porous shell that encompasses the shell, wherein the second shell includes a second set of a plurality of micro- or nanomaterials and interstices formed between the second set of micro- or nanomaterials.

12. The colloidosome of claim 1, wherein the core is a polymer emulsion or a polymer gel, or a void space, and wherein the core is optionally loaded with a core active agent.

13. The colloidosome of claim 1, wherein the overall size of the colloidosome is 5 to 30000 nm, the average size of the plurality of micro- or nanomaterials is 2 nm to 15000 nm, and/or the size of the core is 5 nm to 2500 nm, with the proviso that the size of the core is larger than the average size of the plurality of micro- or nanomaterials.

14. The colloidosome of claim 1, wherein the active agent and/or the core active agent is a chemical agent, a biological agent, an oil, an ionic liquid, a suspension, or a polymer, or any combination thereof.

15. The colloidosome of claim 14, wherein:
the chemical agent is a drug, a cosmetic agent, a flavoring agent, a fragrance-producing chemical, a malodor agent, a reactive agent, a cross-linker, a reactive diluent, a solvent, an inorganic or organic chemical, a metallo-organic system, a petrochemical, a reducing or oxidizing agent, or an aqueous salt, or any combination thereof; and/or
the biological agent is a protein, a peptide, a nucleic acid, a carbohydrate, a lipid, or any combination thereof.

16. The colloidosome of claim 1, wherein the plurality of micro- or nanomaterials are attached to one another by a chemical bond, electrostatic interaction, van der Waals interaction, ionic interaction, hydrogen bonding, dipolar interaction or any combination thereof.

17. The colloidosome of claim 1, wherein the colloidosome is comprised in a pharmaceutical composition, a topical skin care composition, an optically clear medium, or a composition intended to be applied to an inanimate object.

18. A method of using the colloidosomes of claim 1 to deliver an active agent, the method comprising subjecting the colloidosome to a stimulus to release and deliver the active agent.

19. The method of claim 18, wherein the active agent is controllably released from the colloidosome.

20. A method of making the colloidosomes of claim 1, the method comprising:
(a) obtaining a first solution comprising micro- or nanomaterials and a cross-linking agent comprising glutaraldehyde or 1,4-butanediol diglycidyl ether;
(b) obtaining a second solution comprising an active agent; and
(c) combining the solutions to form a colloidosome comprising a micro- or nanostructured porous shell comprising a cross-linked polymeric network;
wherein an active agent is (i) bound to the surface of the micro- or nanostructured porous shell, or loaded or impregnated within the shell of step and/or (ii) present in the liquid medium.
